(19) United States
(12) Patent Application Publication
(10) Pub. No.: US 2004/0260061 A1
(43) Pub. Date: Dec. 23, 2004

(54) Continuous, normal human t-lymphocyte cell lines comprising a recombinant immune receptor with defined antigen specificity

(76) Inventor: Keld Kaltoft, Hammel (DK)

Correspondence Address: BROWDY AND NEIMARK, P.L.L.C., 624 NINTH STREET, NW, SUITE 300, WASHINGTON, DC, 20001-5303, US

(21) Appl. No.: 10/471,481

(22) PCT Filed: Mar. 12, 2002
(86) PCT No.: PCT/DK02/00161

Related U.S. Application Data
(60) Provisional application No. 60/274,643, filed on Mar. 12, 2001.

(30) Foreign Application Priority Data
Mar. 12, 2001 (DK) PA 2001 00415

(51) Int. Cl. C12N 5/08; C07K 14/705

(52) U.S. Cl. 530/350; 435/372

(57) ABSTRACT

The invention describes continuously growing, normal T-lymphocyte cell lines (T-lymphocyte lines) comprising nucleic acids sequences encoding immune receptors, for example T-cell receptors, with defined antigen specificity, wherein the nucleotide sequences are operably linked to an expression signal not natively associated therewith. Further, this invention is directed to methods of adoptive immunotherapy. In particular methods for allogeneic adoptive immunotherapy are provided. This invention further relates to methods for adoptive immunotherapy in the treatment of cancer in a human being, including therapy of malignant melanoma. Moreover this invention relates to methods for cultivating unlimited amounts of activated T-lymphocytes stably expressing a T-cell receptor with defined tumour antigen specificity and reactivity.

This application claims the benefit of U.S. provisional application Ser. No. 60/274,643 filed 12 Mar. 2001 and Danish patent application No. PA 2001 00415 filed 12 march 2002, both of which are hereby incorporated by reference in their entirety. Furthermore, all patent and non-patent references cited in these applications, or in the present application, are also hereby incorporated by reference in their entirety.

### TECHNICAL FIELD

This invention describes continuously growing, normal T-lymphocyte cell lines (T-lymphocyte lines) comprising an immune receptor, including a T-cell receptor, with defined antigen specificity encoded by a nucleotide sequence operably linked to an expression signal not natively associated therewith. Further, this invention is directed to methods of adoptive immunotherapy. In particular methods for allogeneic adoptive immunotherapy are provided. This invention further relates to methods for adoptive immunotherapy in the treatment of cancer in a human being, including therapy of malignant melanoma. Moreover this invention relates to methods for cultivating unlimited amounts of activated T-lymphocytes stably expressing a T-cell receptor with defined tumour antigen specificity and reactivity.

### BACKGROUND OF THE INVENTION

The acquired immune system is based on the combined action of antigen presenting cells (APC) and lymphocytes. It recognises the presence of e.g. a virus at a stage at which viral proteins appear in large amounts in the cytosol. In the cytosol of antigen presenting cells antigenic proteins are first processed by complex proteins termed proteasomes, whose function is to digest polypeptides into shorter peptides. Some of the peptides are then loaded onto one of two classes of major histocompatibility complex (MHC) molecules (in humans also designated human leukocyte antigens, HLA) of antigen presenting cells to be presented to the thymus derived (T) lymphocytes. The T cells recognise the peptide-MHC molecule assemblage via their T-cell receptors and are thereby stimulated to differentiate into effector cells.

T Lymphocytes

The T-lymphocytic lineage is conceived in the bone marrow (fetal liver) and the T lymphocytes are raised and educated in the thymus. In the bone marrow (fetal liver), the same stem cell that gives rise to other blood cells also spawns the progenitor of the T lymphocyte. The progenitor enters the bloodstream, which delivers it to the thymus, and the rest of the development, all the way to the mature T lymphocyte, occurs in this organ.

The mature T cell leaves the thymus by re-entering the bloodstream and spends the rest of its career circulating through the body. The progression from progenitor cell to mature T lymphocyte involves sequential activation or inactivation of groups of genes and the corresponding expression or suppression of their products.

The T-cell Receptor

The function of the T-cell receptor (TCR) is to recognise (foreign) substances (antigens) and to translate the recognition into signals that activates the T lymphocyte. Each T cell expresses approximately 50 000 TCR molecules on its surface but every one of these molecules has the same specificity: they all recognise one particular kind of antigen.

As different T lymphocytes express TCRs of different specifities, the entire pool of T-lymphocytes has the potential of recognising all (foreign) antigens. In addition to the antigen, the TCR also recognises the MHC molecule presenting the antigen on the surface of an antigen-presenting cell. Assisting the receptor proper are the co-receptor and accessory molecules such as CD3.

The antigens recognised by the T-cell receptor are primarily linear peptide sequences bound to MHC molecules. For CD8+ T-lymphocytes (see below) these peptides are usually 8-10 amino acids long. The T-cell receptor recognition of the MHC-antigen complex is usually not as specific as antibody recognition of antigen. Hence, T-lymphocytes are often capable of recognising peptides in the context of an MHC molecule, that differs from one another by one amino acid, albeit with different affinity. In some cases, the natural ligand is not the ligand with the highest affinity. Additionally, the relatively low specificity of the T-cell receptor results in that 2-10% of all T-lymphocytes possess alloreactivity, i.e. they respond to cells of an HLA class not compatible with their T-cell receptor.

The human TCR genes occupy three loci, that are designated TCRA/D, TCRB and TCRG (corresponding to the Greek letters α, δ, β and γ). The first of these three loci is actually a composite of two: the TCRD locus inserted into the middle of the TCRA locus. Two of the four TCR loci (A and G) contain three types of gene segments (V, J and C) and the other two (B and D) contain four (V, D, J and C).

According to the composition of the T-cell receptor (TCR), T lymphocytes fall into two major categories. The four chains of the TCR, α,β,γ and δ can assemble in two combinations (α:β) and (γ:δ). Their expression on the cell surface identifies the α:β T cells and the β:δ T cells. In the mature T lymphocyte pool, α:β T cells constitute the majority and γ:δ T cells the minority.

The antigen specific T-cell receptor (TCR) complex comprises at least 8 polypeptide chains. Two of these chains (the α:β chains) form a disulfide-linked dimer that recognises antigenic peptides bound to MHC molecules. These chains are the actual ligand-binding structure within the TCR. The amino-terminal regions of the α and β chains are highly polymorphic, so that within the entire T-lymphocyte population there are a large number of different TCR α/β dimers, each capable of recognising or binding a particular combination of antigenic peptide and MHC.

The α/β dimer associates with the CD3 complex, which is composed of the γε, δε and ξξ pairs. T-lymphocyte activation is triggered by ligation of the TCR with the peptide-MHC molecule assemblies on the surface of the APC. According to one presently preferred hypothesis, ligation presumably includes aggregation of TCR complexes and thus brings together intracellular CD3-associated protein kinases, enabling them to phosphorylate each other as well as the intracellular domains of the ξ chains. Simultaneously, coligation of the co-receptors CD4 or CD8 (depending on the nature of the MHC molecule involved) results in the phosphorylation of the co-receptor-associated Lck kinases.

The Diversity of T-Cell Receptors

To generate the diversity of TCRs, required to recognise a wide spectrum of antigenic determinants, the TCR α and β genes use a combinatorial strategy of DNA rearrangement similar to that of the immunoglobulin genes. The germline TCR β gene contains about 65 V (variable), 2 D (diversity), 13 J (joining) gene segments and 2 C (constant regions segments).

When the TCR β gene rearranges early in T-lymphocyte development, one of the V_{β} region segments becomes linked to one of the D_{β} regions and to one of the J_{β} segments to form a single transcriptional unit. The V-D-J splices to a constant C_{β} (constant) region to form a TCR β mRNA that encodes a functional protein. Great diversity is generated by this combinatorial joining. In the TCR α locus, there are more than 45-50 segments V segments and about 60 J segments, one C segment and no D segments. To form a functional TCR α chain gene, a V_{α} segment joins to a J_{α} segment and the V-J transcript splices to a constant region (Cα).

Diversity is further enhanced by imprecise joining of the gene segments and/or by the insertion of non-germline-encoded nucleotides (designated N regions) between segments during the rearrangement process. These mechanisms generate junctional diversity, in particular the diversity of sequences at the junctions between V_{α} and J_{α} and between V_{β}, D_{β}, and J_{β} segments. The V-J and V-D-J junctional sequences are unique to each T-cell receptor clonotype and contribute to the T-cell receptor diversity.

Helper and Cytotoxic T-Lymphocytes

Based on their function, α:β T lymhocytes can be divided into at least two subsets, helper T cells and cytotoxic T cells. Helper T cells (T_{H}) are so designated because, upon activation, they secrete a number of cytokines that control and coordinate other cells participating in the ongoing immune response. TH are CD4+CD8− and recognise antigen in the context of class II MHC molecules. They constitute about 60% of mature T-lymphocytes.

Cytotoxic T lymphocytes (CTLs), when activated, acquire the capacity to lyse target cells carrying antigens recognised by their TCR. Cytotoxic T-lymphocytes are CD4−CD8+ and MHC class I restricted. The γ:δ cells can also recognise the antigen alone, without the participation of MHC molecules.

Conventional Adoptive Immunotherapy Methods Exploiting Polyclonal Tumour Infiltrating Lymphocytes and Cloned Tumour Infiltrating Lymphocytes

Many human tumours express tumour associated antigens and the existence of tumour reactive lymphocytes strongly suggests that an immune recognition to cancer exist in humans. However, although immune recognition to cancer exist ample evidence demonstrates that immune recognition is necessary but not sufficient for generating an immune response. As will be apparent from the below summary of state of the art of adoptive immunotherapies exploiting tumour infiltrating lymphocytes (TIL), such therapies are not sufficiently effective against a large number of human cancers.

Several protocols depending on the adoptive transfer of TILs have been tested. The problems associated with employing TILs in cancer therapy are described in the following sections.

By means of the T lymphocyte growth factor interleukin-2 (IL-2) it is possible in the laboratory to expand TIL's and to transfer these cultured cells back to the patient. However, the culture protocols aimed at expanding TIL's are timely and select for the fastest growing T lymphocytes, but not necessarily for T lymphocytes with tumour specificity.

When a sufficiently large tumour specimen comprising at least 1-2 g lymphocytes is available, it is possible to culture ˜10¹¹ lymphocytes within 2 months using conventional protocols. However, this is only the case in approx. 50% of cancer patients and unfortunately, only a minor fraction of these lymphocytes are tumour specific and the vast majority of nonspecific lymphocytes are of no therapeutic value.

The present inventor has demonstrated that use of conventional TIL culture protocols results in a selection against melanoma specific T lymphocytes upon long term culture. These experiments agree with other studies showing that younger TIL cultures contain a higher fraction of tumour specific T-lymphocytes, as compared to long term TIL cell cultures (Schwartzentruber et al., 1994, Arienti et al., 1993, Rosenberg et al., 1994). Also, a culture period of approximately 2 months needed to expand the TIL's in conventional protocols has the disadvantage that disease progression may occur before the immune therapy can be initiated.

Additionally, several phase 1 and 11 protocols combining TIL therapy with IL-2 and/or chemotherapy have been performed (Goedegebuure et al., 1995, Schwartzentruber et al., 1994, Arienti et al., 1993, Rosenberg et al., 1994, Queirolo et al., 1999). Unfortunately, the results of these experiments are not significantly better than what can be obtained by IL-2 therapy alone and it is therefore important to develop better protocols for adoptive immunotherapy.

The reason for the disappointing results with adoptive immunotherapy may result from the fact, that only relatively few tumour specific TIL's reach the tumour. In particular, the number reaching the tumour is not sufficient for generating adequate tumour cell killing and a satisfactory cytokine/chemokine production.

An intrinsic cytokine production of T-lymphocytes is desirable as an inappropriate cytokine production leads to the immune system of the patient not being sufficiently activated to combat the disease.

Although the secretion of IFN-γ by injected TILs have been shown to significantly correlate with in vivo regression of murine tumours (Barth et al., 1991), it has generally been difficult to obtain T-lymphocytes with a desirable, long lasting cytokine expression profile.

For finite human T lymphocytes, the best estimate of replicative senescence is 23 PD (Perillo et al., 1989). This means that one T cell on average can generate 2²³=10⁷ (approximately 10 milligram) T lymphocytes. Although such T cell clones may be tumour specific, this number of T cells is not considered to be sufficient for immunotherapeutic trials even if several clones can be cultured from the patient (Dunbar et al., 1999). Due to the relative small number of tumour specific T lymphocytes it is furthermore only possible to treat the patient from which the T lymphocytes derive (autologous adoptive immunotherapy).

Intravenous injection of cultured TIL's has been applied in most immunotherapeutic protocols. Such transfer of TIL's leads to accumulation of the transferred lymphocytes in the lung capillaries resulting in the death of most of the TIL's after 2-3 days. A small fraction of the surviving TIL's then migrates and accumulates preferentially in tumour tissue. This implies that only a minor fraction of the infused T lymphocytes reach tumour tissue and of those that home to the tumour bed only a small proportion has tumour specificity. Hence, this approach is not effective in clinical terms.

Another concern using cultured T cells is that they have to retain their functions in vivo in order to be useful in adoptive immunotherapy. In particular, it has been observed that antigen-specific T cells which were grown long term in culture in high concentrations of IL-2 may develop cell cycle abnormalities and lose the ability to return to a quiescent phase when IL-2 is withdrawn. T lymphocytes that are exposed to high concentrations of IL-2 to promote cell growth will often die by a process called apoptosis if IL-2 is removed, or if they are subsequently stimulated through the T cell receptor, i.e., if they encounter specific antigens. (Lenardo, 1991).

To circumvent this problem many adoptive immunotherapy protocols have included systemic treatment with IL-2. However infusion of IL-2 is associated with extreme toxicity. Described side effects of systemic IL-2 treatment are for example hypotension due to leaky cappilary syndrome, fever with chills, nauseas and vomiting, diarrhea, cutaneous rashes with eythema and dermal vascularitis. Renal failure and edema have also been observed. It is therefore desirable to omit systemic administration of IL-2.

Malignant Melanoma

Malignant melanoma is one type of cancer in humans against which no effective method of treatment currently exists. Malignant melanoma make up a serious health problem and the incidence has increased worldwide during the last decades. Alone in Denmark with a population of 5 million there are approximately 900 new cases pr. year. Most patients can be cured by surgery, but 10-20% corresponding to approx. 100 to 200 individuals per year, will either show disseminated disease at the time of diagnosis or will after surgery develop metastatic disease. The prognosis for patients with metastatic disease is in general very poor with a median survival time between 4,4 and 12.5 months (Barth et al., 1995). Untreated, the two year survival rate is less than 5%. WHO estimates that malignant melanoma was responsible for 6,000 deaths in the Americas and 12,000 deaths in Europe in 1999.

Malignant melanoma is characterised by an infiltration among the tumour cells of cells (leukocytes) of the immune system, for instance T lymphocytes. It has been known for a long time that some of these tumour infiltrating lymphocytes (TILs) have specificity directed against the tumour cells. Specificity is in general monitored in the laboratory by activating TIL's with IL-2. Specificity is not alone sufficient for tumour cell killing activity and cytokine production. Unless specific T cells are further activated/costimulated no reactivity (meaning killing and cytokine production) against the tumour cells occurs. As malignant melanoma progresses rapidly it is apparent that these TIL's in vivo do not have sufficient reactivity and in vivo they are also not capable of efficiently activating other effector cells against the melanoma (Goedegebuure et al., 1995). TILs have been isolated from metastatic melanoma where they recognise melanocyte-melanoma lineage specific tissue antigens in vitro and in vivo, e.g. Gp100, MART-1 and tyrosinase. (Kawakami et al., 1993), Anichini et al., 1993). The data implies that although TIL's have melanoma specific T cell receptors that are able to bind to melanoma associated antigens these TIL's do not in vivo have sufficient reactivity to combat the tumour. Apparently TIL's suffer from anergi, most likely due to insufficient activation and/or costimulation.

WO 96/30516 (Nishimura) describes nucleic acid sequences for T-cell receptors which recognise tumour associated antigens. In particular, T-cell receptors which recognise melanoma antigens are described, but not in combination with normal, continuous T-lymphocyte lines. WO 96/30516 also describes thymocytes in the form of Jurkat cells expressing the antigen specific T-cell receptors. However, the thymocytes are of malignant origin. In addition, WO 96/30516 provides stem cells expressing the antigen specific T-cell receptors or chimeric receptors. Stem cells are not normal, continuous T-lymphocyte lines. WO 96/30516 further relates to therapeutic and diagnostic compositions and methods employing the T-cell receptors and chimeric receptors.

α and β chains of a T-cell receptor specific for tumour antigens have been cloned and transduced into either i) a Jurkat cell (WO 96/30516; Liu et al., 2000), which is a thymocyte cell line without any cytotoxic activity which is derived from an acute T-lymphocyte leukemia, or ii) a murine bone marrow progenitor cells, for example MART-1 specific TCR or p53 specific TCR (Liu et al., 2000). Jurkat cells comprising MART-1 specific TCR do not recognise tumour cells expressing HLA-A2 and MART-1, and the encounter does not lead to cytokine production (WO 96/30516). The present invention does neither pertain to Jurkat cells, or any other cell line of malignant origin, nor to bone marrow progenitor cells.

### SUMMARY OF THE INVENTION

There is a need for i) solving the problems associated with the state of the art methods for treatment of human cancers, and ii) developing novel strategies for exploitation of antigen specific, tumour reactive lymphocytes in adoptive immunotherapies.

In this respect, it is of paramount importance for effective adoptive immunotherapy of cancer to be able to isolate and expand antigen-specific T cells in large numbers by in vitro culture and to ensure that these cultured T-lymphocytes, following adoptive transfer, retain their antigen specificity and persist and function in vivo. Further, it is important that the time required to culture the T-lymphocytes is limited to prevent undesirable progression of disease.

T-Lymphocytes Comprising Antigen Specific Immune Receptors Including T Cell Receptors

It is one objective of the present invention to provide a method for treatment of a clinical condition in an individual, including a human being, by administrering to said individual a normal T-lymphocyte cell line comprising an antigen specific immune receptor encoded by a nucleotide sequence operably linked to an expression signal not natively associated therewith, wherein the amount and/or presence of antigen is indicative of the occurrence of the clinical condition.

The clinical condition could for example be a cancer or a viral infection.

It is another objective of the present invention to provide continuous, normal T-lymphocyte cell lines, which are preferably continuous, normal, human T-lymphocyte cell lines comprising an antigen specific immune receptor encoded by a nucleotide sequence operably linked to an expression signal not natively associated therewith. Preferably, such T-lymphocytes are used in the above method of treatment and capable of being administered to an individual suffering from said clinical condition.

Accordingly, when the clinical condition is a cancer, the cancer specific immune receptor recombinantly expressed by the T-lymphocyte according to the invention has an affinity for at least one antigen associated with said cancer.

It is yet another objective to provide continuous T-lymphocyte cell lines comprising nucleic-acids encoding an antigen specific immune receptor with defined specificity, wherein said T-lymphocytes further comprises a predetermined intrinsic cytokine production.

The therapeutic method of treatment is preferably a prophylactic and/or curative and/or ameliorating and/or palliative therapeutic method, wherein said T-lymphocytes comprising said nucleic acid sequences encoding antigen specific immune receptors, including T-cell receptors, capable of recognising a tumour associated antigen are administered in pharmaceutically effective amounts to an individual in need of such administration.

In particular, it is an object of this invention to methods of treatment which are allogeneic immunotherapy employing said T-lymphocytes comprising said nucleic acid sequences, said methods comprising said T-lymphocytes comprising said nucleic acid sequences.

The continuous, normal T-lymphocyte cell line is preferably established by

(a) cultivating, in vivo and/or in vitro, activated T-lymphocytes to continuous growth in the presence of high concentrations of at least two factors promoting T-lymphocyte growth, preferably in the presence of high concentrations of at least two cytokines including IL-2 and IL-4, thereby establishing at least one normal T-lymphocyte cell line; and

(b) introducing into said normal T-lymphocyte cell line at least one nucleic acid sequence encoding at least one antigen specific immune receptor, or a part thereof, operably linked to an expression signal not natively associated therewith, and

(c) thereby obtaining a continuous, normal T-lymphocyte cell line comprising said nucleic acid sequences.

One method for cultivation of continuous T-lymphocytes is described in WO 99/00363, which is incorporated herein by reference in its entirety for all purposes. In a preferred embodiment of the present invention the T-lymphocytes is a normal, human T-lymphocyte cell line.

It is generally preferred, that the T-lymphocytes are capable of expressing the at least one antigen specific immune receptor encoded by said nucleic acid sequence(s). Hence, the expression signal should be selected so that it allows expression in T-lymphocyte cell lines, preferably in human T-lymphocyte cell lines.

Furthermore, it is preferred that the T-lymphocyte cell lines are capable of continuously expressing the at least one antigen specific immune receptor. In addition it is preferred that the at least one antigen specific immune receptor encoded by said nucleic acid sequence(s) is capable of inducing appropriate signal transduction resulting in T-lymphocyte reactivity.

Previously, it has been difficult to obtain stable expression of transgenic antigen specific immune receptors in human T-lymphocytes. In particular, it has been difficult to obtain T-lymphocytes stably expressing a transgenic antigen specific immune receptor, wherein said stable expression have resulted in T-lymphocytes that continuously are capable of inducing reactivity upon encounter with the specific antigen (McInerney et al, 2000, Rossig et al., 2001, Kessels et al., 2001 and Liu and Rosenberg 2001).

However, the present invention provides human T-lymphocyte cell lines capable of stably expressing a transgene. In addition the present invention provides human T-lymphocyte cell lines capable of stably expressing an antigen specific immune receptor, wherein the T-lymphocyte cell lines are capable of inducing reactivity upon encouter with the specific antigen.

In particular, the at least one nucleic acid encoding the at least one antigen specific immune receptor is preferably selected from nucleic acid sequences encoding an antigen specific T-cell receptor.

Preferably, the at least one nucleic acid encoding the at least one antigen specific immune receptor is selected from nucleic acid sequences encoding Variable-Joining (V/J) sequences of an α chain or Variable-Diversity-Joining (V/D/J) sequences of a β chain of an antigen specific T-cell receptor. In particular, the invention relates to nucleotide sequences encoding a T-cell receptor as described herein.

This invention further relates to pharmaceutical compositions comprising at least one active ingredient in the form of a T-lymphocyte cell line according to the present invention comprising at least one nucleic acid sequence encoding an antigen specific immune receptor.

It is yet another objective of the present invention to provide the use of a continuous, normal T-lymphocyte cell line comprising an antigen specific immune receptor encoded by a nucleotide sequence operably linked to an expression signal not natively associated therewith in the manufacture of a medicament for treatment of a clinical condition in an individual, including a human being, wherein the antigen for which the immune receptor is specific is indicative of the occurrence of the clinical condition in the individual.

It is another object of the present invention that T-lymphocytes for adoptive immunotherapy, preferably against cancer, should have cytotoxic activity against diseased cells expressing an antigen recognised by the specific immune receptor. In case of treatment of tumours, the T-lymphocytes should preferably have tumour cell killing activity. In another preferred embodiment, this invention relates to continuous T-lymphocyte cell lines comprising nucleic acids encoding an immune receptor with defined specificity, said T-lymphocytes having cytotoxic activity.

Furthermore, activated T-lymphocytes with a pre-determined intrinsic cytokine production are preferred, as cytokines are capable of activating the endogenous cells of the immunesystem.

It is another object of the present invention to provide a method of cultivating the continuous T-lymphocyte cell lines comprising antigen specific immune receptors of the present invention, said method comprising the steps of:

i) Providing said T-lymphocyte cell line

ii) Cultivating said T-lymphocyte cell line under conditions allowing expression of the antigen specific immune receptor.

### LEGENDS TO FIGURES

FIG. 1 illustrates the scenario that at best may occur upon injection of the T-lymphocytes of the present invention directly into a tumour.

FIG. 2 illustrates the retroviral expression vector encoding the A7 T-cell receptor. LTR stands for long terminal repeat of Moline murine leukemia virus, α and β-chain are the α (the α-chain belongs to the α1.1 subfamily T cell receptor) and β-chain (the β-chain belongs to the β 7.3 subfamily of the T cell receptor) of the melanoma specific A7 T cell receptor. Expression of the T cell receptor α-chain is driven by the 5′LTR promoter, which also drives the expression of the neomycin phosphotransferase gene (neo) via an internal ribosomal entry site (IRES). The β-chain expression is driven by a hybrid HTLV-I/SV40 SRα promoter. Ψ⁺ is a packaging signal, SD splice donor site, SA splice acceptor site and pA polyadenylation site. The arrows shows the transcription sites.

FIG. 3 illustrates T-lymphocyte transfection and tumour cell/cytotoxic T-lymphocyte interaction. A) genes encoding a Mart-1 specific T-cell receptor. Cytokine release includes release of IFN-γ, TNF-α, GM-CSF and IL-5.

FIG. 4 illustrates a flow cytometric analysis of the common phenotypic markers of C-Cure 707 and C-Cure 709.

FIG. 5 illustrates T cell receptor expression of C-Cure 707 and C-Cure 709 over time.

FIG. 6 illustrates specific recognition of Mart-1 (M9-2) by C-Cure 709 as measured by induction in IFN-γ production.

### DEFINITIONS

For the purpose of a more complete understanding of the invention, the following definitions are described herein.

Activated T-lymphocytes: T-lymphocytes wherein a signal has been induced by an external influence. Such influence could for example be recognition by the T-cell receptor of one or more antigens, either in the context of an antigen presenting cells or as an isolated MHC/antigen complex. Examples of antigens is tumour associated antigen(s), that could also be presented by a tumour cell, viral antigen(s), alloantigen(s), or super-antigen(s). Super-antigens could be SEA, SEB, SEC, SED, SEE, TSST, Streptococcus pyogenes enterotoxin A, B and C, and Mycoplasma arthritidis antigen. Alternatively, such influence could be the presence of a cytokine, such as IL-2 and/or IL-4 or a mitogen such as PHA and jacalin. Futhermore, T-lymphocytes can be activated by antibodies towards CD2, CD3, CD28 and/or TCR or by addition of ionomycin, phorbolester and/or TPA. T-lymphocytes can also be activated by allostimulation with appropriate allogeneic cells. Furthermore, the activation could be accomplished by a combination of any of the above mentioned influences.

Adoptive immunotherapy: Therapy comprising administration of in vitro expanded lymphocytes to a patient.

Allogeneic adotive immunotherapy: Therapy comprising administration of in vitro cultivated lymphocytes to a patient, said lymphocytes being derived from an individual other than the patient.

Continuous T-lymphocyte line: Any T-lymphocyte line including a normal T-lymphocyte line capable of having an in vitro life span of at least 30 population doublings (PD), such as at least 40 PD (i.e. 1 cell becoming approximately 1 kg. of cell mass), such as at least 60 PD (i.e. 1 cell becoming approximately 1000 tons of cell mass), such as at least 80 PD, preferably at least 100 PD, more preferably at least 150 PD, such as at least 200 PD. The term continuous T-lymphocyte line further pertains to T-lymphocytes wherein the functional profile are not substantially altered during the continuous growth, meaning that the function of the T-lymphocytes essentially correspond to the initial cells. In certain cases, re-activation with any one or more of one or more antigens, one or more antibodies, one or more super antigens and/or any chemical compound capable of activating the T-lymphocyte may be used to activate the T-lymphocytes to an increased growth rate, phenotypical and functional integrity, such as increased cytokine production.

Disease activated T-lymphocytes: T-lymphocytes wherein a signal has been induced by an external influence, wherein said external influence is the result of a disease of the individual comprising said T-lymphocytes.

Expression control sequence: A sequence that is conventionally used to effect expression of a gene that encodes a polypeptide and include one or more components that affect expression, including transcription and translation signals. Such a sequence includes, for example, one or more of the following: a promoter sequence, an enhancer sequence, an upstream activation sequence, a downstream termination sequence, a polyadenylation sequence, mRNA ribosomal binding sites, an optimal 5′ leader sequence to optimise initiation of translation in mammalian cells, a Kozak sequence, which identifies optimal residues around initiator AUG for mammalian cells and/or a translation termination sequence.

Factors which promote T-lymphocyte growth: Includes any biological and/or chemical compound, cell and the like which directly and/or indirectly stimulate T-lymphocyte growth (see below).

Inflammation: Local accumulation of fluid, plasma proteins, and white blood cells that is initiated by physical injury, infection, or a local immune response. This is also known as an inflammatory response. Acute inflammation is the term used to describe transient episodes, whereas chronic inflammation occurs when the infection persists or during auto-immune responses. Many different forms of inflammation are seen in different diseases. The cells that invade tissues undergoing inflammatory responses are often called inflammatory cells or an inflammatory infiltrate.

Intrinsic cytokine production: T-lymphocytes produce and secrete, one or more cytokines which could be selected from, but is not limited to: IL-2, IL-4, IL-5, IL12, IFN-γ, TNF-α, GM-CSF, C-CSF either constantly or after activation.

Major Histocompatibility Complex (MHC): A generic designation meant to encompass the histocompatibility antigen systems described in different species including the human leucocyte antigens (HLA).

Melanoma includes, but is not limited to, melanomas, metastatic melanomas, melanomas derived from either melanocytes or melanocyte related nevus cells, melanocarcinomas, melanoepitheliomas, melanosarcomas, occular melanoma, melanoma in situ, superficial spreading melanoma, nodular melanoma, lentigo maligna melanoma, acral lentiginous melanoma, invasive melanoma or familial atypical mole and melanoma (FAM-M) syndrome.

Nucleic acid sequences include, but are not limited to, DNA, RNA, cDNA, PNA and LNA.

Normal T-lymphocyte line: Non-malignant T-lymphocyte line that is of non-malignant origin.

Pharmaceutically effective amount: An amount sufficient to induce a desired biological result. The result can be alleviation of the signs, symptoms, or causes of a disease, or any other desired alteration of a biological system, including tumour regression. For example, an effective amount is generally that which provides either subjective relief of symptoms or an objectively identifiable improvement as noted by the clinician or other qualified observer. In particular, such an effective amount results in reduction of tumour cell mass. Accordingly, effective amounts can vary widely depending on the individual, on the disease or symptom to be treated.

Substantially homologous nucleic acids: Substantial correspondence between the nucleic acid sequence for the V-J or V-D-J junctional sequences for the α and β chains of the tumour antigen specific T-cell receptors provided herein and that of any other nucleic acid sequence. By way of example, substantially homologous means about 80-100% homology, preferably by about 85-100% homology, and more preferably about 90-100% homology, and most preferably about 95-100% homology, such as 98-100% homology, between the nucleic acid sequences and that of any other nucleic acid sequence. In addition, substantially homologous as used herein also refers to substantial homologies between the amino acid sequence of the V-J or V-D-J junctional sequences of the antigen specific T-cell receptors provided herein and that of any other amino acid sequence.

“T-lymphocyte” and “T-cell” are used interchangeably herein.

T-cell reactivity: The kind of reactivity of a specific T-cell is dependent on the kind of T-cell. For cytotoxic T-cells, T-cell reactivity normally is initiated by specific antigen recognition and may include cytotoxic activity and cytokine production. Hence, reactivity of cytoxic T-cells may for example be determined by assays measuring cytotoxic activity of said cytoxic T-cells and/or by assays measuring cytokine production of said T-cell. In particular, T-cell reactivity may be initiated by antigen recognition by an antigen specific immune receptor expressed by said T-cell, for example the antigen specific immune receptor may be a Tell receptor.

Tumour associated antigen(s)/cancer associated antigen(s): Comprise any antigen(s) (foreign or auto-antigen(s)) that are associated with a tumour which for example can be selected from: melanoma, epithelial cell derived cancers, lung cancer, colon cancer, ovarian cancer, breast cancer, kidney cancer, prostate cancer, brain cancer, Sezary's syndrome, lymphoma, leukemia, cancer of the uterus, hepabc carcinoma or sarcomas

Tumour/cancer: Includes but is not limited to, melanoma, carcinoma, lung cancer, colon cancer, ovarian cancer, breast cancer, kidney cancer, prostate cancer, brain cancer, lymphomas, leukemia, Sezary's syndrome, cutaneous T-lymphocyte lymphoma, cancer of the uterus, hepatic carcinoma or sarcomas. Such cancers in mammals may be caused by, chromosomal abnormalities, degenerative growth and developmental disorders, mitogenic agents, irradiation, viral infections, inappropriate tissue expression of a gene, alterations in expression of a gene, or carcinogenic agents.

### DETAILED DESCRIPTION OF THE INVENTION

Adoptive Immunotherapy Against Cancer

It is one objective of the present invention to provide a method for prophylactic or therapeutic treatment of cancers. Therapeutic treatment should be understood as both curative and/or ameliorating and/or palliative treatment. In particular it is an object of this invention to provide continuous, normal T-lymphocyte cell lines comprising receptors that recognise cancer associated antigens for use in allogeneic adoptive immunotherapy. In a preferred embodiment the T-cell receptor is specific towards a melanoma specific antigen, such as a malignant melanoma specific antigen.

Examples of cancerous diseases which could be treated with the T-lymphocyte lines prepared according to the present invention include, but is not restricted to malignant melanoma, renal carcinoma, breast cancer, lung cancer, cancer of the uterus, prostatic cancer, lymphoma, leukemia, cutaneous lymphoma, hepatic carcinoma, colorectal cancer and sarcoma.

In addition, it is another object of this invention to provide a method of treatment that is a combination therapies against cancer comprising said T-lymphocytes comprising said nucleic acids combined with one or more different cancer therapies which could be selected from: surgical treatment, chemotherapy, radiation therapy, therapy with cytokines, Hormone therapy, gene therapy, dendritic cell therapy or treatments using laser light.

Chemotherapy could include therapy using one or more drugs selected from: Melphalan, Carboplatin, Cyclophosphamid, Cisplatin, Ifosfamid, Chiorambucil, Lomustin Treosulfan, Temozolomid, Cytarabin, Azathioprin, Metothrexat, Fludarabinphosphat, Fluoruracil, Gemcitabin, Azathioprin, Cladribin, Podophyllotoksin, Etoposid, Topotecan, Vinkristin, Paclitaxel, Docetaxel, Vinblastin, Etoposid, Teniposid, Aclarubicin, Doxorubicin, Doxorubicin, Mitomycin, Mitoxantron, Idarubicin, Anon, Lenograstin, Filgrastim, Aldesleukin, Verteporfin, epirubicin, daunorubicin, valrubicin and adriamycinon.

Cytokine therapy could be therapy using one or more cytokines selected from, but not limited to: IL-2, IL-4, IL-10, IL-12, IL-15, IL-18, IL-21, IFN-γ, IFN-α, GM-CSF, C-CSF.

Dendritic cell therapy could be any immunotherapy based on dendritic cells. Immunotherapy based on dendritic cells has recently attracted broad attention due to the fact that it is now possible to culture pure dendritic cells in the laboratory. One method for cultivating dendritic cells could be for 8-12 days in a medium containing GM-CSF and IL-4, often adding TNF-α at the end of the culture period, however any other protocol known to the person skilled in the art may be applied. Following cultivation, the dendritic cells can be pulsed with tumour associated peptides or tumour cell lysates and injected into the patient. Injection could be either directly into the tumour or into an uninvolved lymph node or any other suitable way of injection.

In one embodiment of dendritic cell therapy to be used in combination with the herein disclosed invention, the potency of dendritic cells serving as antigen presenting cells is further strengthened by genetic manipulation. For example this could be insertion of cytokine genes, genes coding for tumour associated antigens or any other gene encoding proteins known to influence the immune system.

In one preferred embodiment the T-lymphocytes of the present invention are used in combination with a dendritic cell therapy as described herein below. This therapy is especially usefull for treating malignant melanoma and has been developed by the company Bavarian Nordic. The gene encoding human tyrosinase has been inserted into the aftennuated smallpox derivative MFA-F6 (Modified Vaccinia Ankara F6) with the intention of infecting-cultured dendritic cells. In particular, it is intended to infect cultured autologous dendritic cells with this construct. These autologous dendritic cells will then overexpress the tyrosinase gene and due to their potent antigen presenting capacity, they are expected to mount an immune response against a melanoma upon transfer to a patient. This approach is expected to provoke the immune danger signals that maximally turns the immune system against the melanoma.

Although IL-2 treatment may be desirable under some circumstances, it is a preferred objective of the present invention to provide treatment wherein the continuous T-lymphocytes lines of the present invention comprising antigen specific immune receptors are administrated without simultaneous systemic IL-2 treatment.

In one preferred embodiment the T lymphocytes according to the present invention are able both directly and indirectly to activate cells of the immune system.

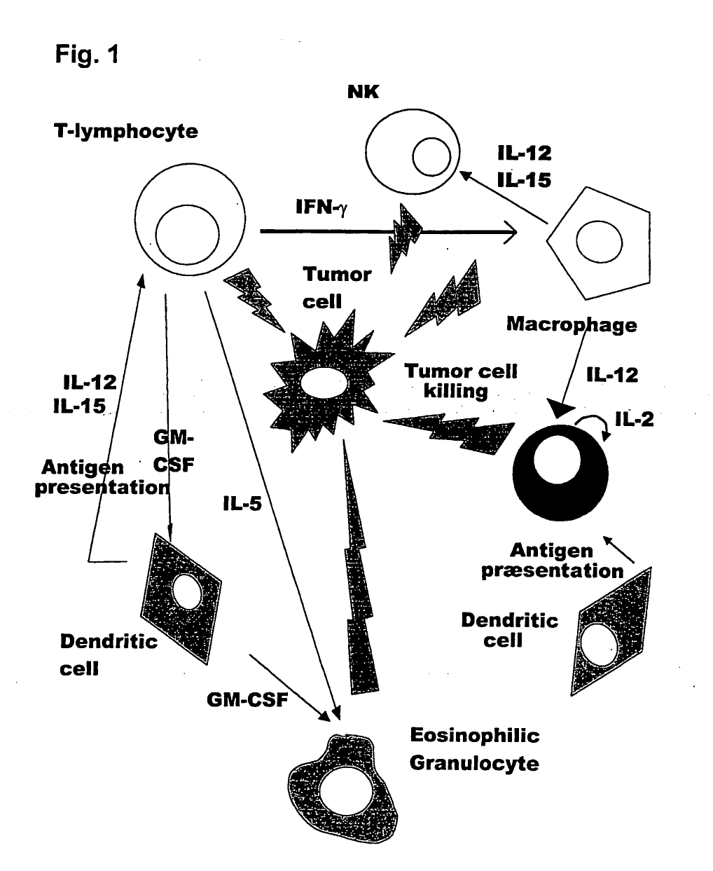

Preferably, the T lymphocytes of the present invention comprising antigen specific immune receptors are administrated by direct injection of a pharmaceutical composition comprising an effective amount of said T-lymphocytes into the edge of a tumour tissue. It is preferred that the following scenario occurs after such administration (see FIG. 1): The T-lymphocytes comprising antigen specific immune receptors recognise tumour cells expressing the specific antigen. In the case that the immune receptor is a T-cell receptor, the T-lymphocytes recognise tumour cells expressing the specific antigen in the context of a MHC molecule of a subclass recognised by the T-cell receptor.

More preferably, in case of low expression of the antigen/MHC complex, constitutive production and secretion of IFN-γ by the T-lymphocyte cell lines of the present invention are expected to upregulate MHC expression and to induce heat shock proteins that render tumour cells more immunogenic. Even more preferably, IFN-γ secreted by said T-lymphocytes activate resident macrophages and NK cells to cytokine production and non-specific tumour cell killing activity. Activated NK cells are known to kill tumour cells with low or no HLA class I expression (Hung et al, 1998).

Preferably, activation of macrophages (see FIG. 1) leads to production of a number of cytokines among others GM-CSF. Macrophage derived GM-CSF Will together with high production of GM-CSF from the T-lymphocytes of the present invention lead to an influx of immune cells, for instance dendritic cells.

Preferably, after activation dendritic cells will ingest apoptotic bodies and necrotic tumour cell debris resulting from the tumour cell killing activity. It is furthermore preferred that the dendritic cells act as potent antigen presenting cells and thereby activate autologous resident tumour specific T lymphocytes (FIG. 1). This leads to improved tumour recognition and preferably generate activated T lymphocytes recognising tumour-associated antigens different from the one recognised by the immune receptor of the present invention.

It is a further preferred object of the present invention that activated dendritic cells and macrophages produce IL-12 and IL-15-that activate the T-lymphocytes of the present invention as well as autologous immune cells.

It has previously been shown that GM-CSF production by TIL's is associated with treatment efficiency by clinical trials (Schwarzentruber et al, 1994). Further it has been shown that vaccination of malignant melanoma patients with γ-irradiated GM-CSF producing melanoma cells (GVAX vaccine) is followed by an influx to the tumour metastasis of leukocytes (Soiffer et al, 1998).

Preferably, the T-lymphocytes of the present invention produce and secrete IL-5, that activates eosinophilic granulocytes that efficiently participates in non-specific tumour cell killing activity (Hung et al., 1998).

Preferably, activation of tumour specific T lymphocytes, either CD4+ and CD8+ T lymphocytes, lead to IL-2 production. The produced IL-2 preferably, stimulate resident T cells to tumouricidal activity as well as increase the tumouricidalactivity of the T-lymphocytes of the present invention.

Preferably, tumour-associated antigens are presented to autologous T lymphocytes even if there is low or no MHC expression on the tumour cells. This is, as discussed above achieved due to antigen presentation by dendritic cells, a phenomenon known as cross priming or cross presentation.

Preferably, direct injection of the T-lymphocytes of the present invention into the edge of tumour tissue, generates a “bridgehead” where the autoimmune process leading to tumour cell destruction is optimal. More preferably, the concentration of injected T-lymphocytes is high, because a fraction of these cells as well as autologous T cells are expected to die upon recognition and activation by the tumour cells. The killing of tumour cells thus results in suicide of activated T lymphocytes, a phenomenon known as activation induced cell death (AICD). Most preferably, AICD is prevented by simultaneous administration of caspase inhibitors (Zaks et al., 1999).

Activation induced cell death (AICD) is expected to lead to elimination of the administered lymphocytes, which in general should be sensitive to Fas-FasL killing in order not gain access to immune privileged sites such as the eyes and the testis.

Preferably, the high concentration of injected T-lymphocytes of the present invention combined with the influx of macrophages, dendritic cells, NK cells and eosinophilic granulocytes will soon establish an environment in which antigen presentation to autologous T lymphocytes primarily will occur by dendritic cells. The consequence of this will be good costimulation of T lymphocytes and diminished AICD. More preferably, the autologous T lymphocytes proliferate and by their homing markers migrate to other metastatic tumour sites It is furthermore a preferred embodiment that chemokine and cytokine production is centred in the tumour, thus avoiding the severe side effects of systemic IL-2 treatment as often observed in TIL trials. More preferably, the cytokine production of the T-lymphocytes of the present invention will be ongoing for 1-2 days. This is contrary to systemic IL-2 treatment where the infused IL-2 is broken down quickly.

The injection of the T-lymphocytes of the present invention is thus expected to start a potent autoimmune cascade primarily directed against the tumour. Besides the T-lymphocytes of the present invention a number of autologous leukocytes are activated hereby, establishing collateral tumour cell damage. If the autoimmune process is of sufficient strength the autologous activated immune cells are expected to migrate from the injected tumour tissue to metastasis overall in-the body. This may at best lead to complete remission.

In one preferred embodiment the T-lymphocytes of the present invention are capable of killing tumour cells. However, it should be pointed out that although the tumour cell killing of the T-lymphocytes of the present invention is important the major task of the T-lymphocytes of the present invention is efficiently to activate the immune system of the patient against the tumour. The inflammatory T-lymphocyte cell lines of the present invention have this property, because of their constitutive and inducible cytokine production.

The advantages of the T-lymphocytes of the present invention compared to TIL's are several: Firstly, said T-lymphocytes are a specific inflammatory continuous T cell line which can be used world wide as an “off the shelf” pharmaceutical. Furthermore treatment can start immediately after diagnosis, HLA typing arid test for the presence of the specific antigen. The side effects are also expected to be milder than systemic IL-2 treatment. Finally, It is also feasible to culture said T-lymphocytes in GLP/GMP facilities in serum free medium making it possible to register them as an approved pharmaceutical.

In particular, this invention enables the treatment of patients that are HLA compatible with the immune receptor comprised within the T-lymphocytes of the invention. HLA-typing may for example be performed on peripheral blood cells, biopsies or the like. Although it is preferable to treat tumours that are HLA compatible with said immune receptor and that express the antigen recognised by said immune receptor, the T-lymphocytes of comprising said immune receptors could also be used for treating other tumours, due to the preferably long lasting cytokine production of such T-lymphocytes.

A host versus graft reaction is expected 10-14 days following the first administration of T-lymphocytes to a patient, as this can be regarded as an allogeneic transplantation. In general this reaction will most likely primarily be directed against HLA class I and II antigens on the T-lymphocytes according to the present invention.

Besides these immunodominant molecules the recombinant immune receptor and selection markers are immunogenic, as they are neo-antigens. Other allogeneic differences between donor and graft can of course also give rise to a host versus graft reaction. These immune reactions are not expected to provoke severe side effects, because of the relative low amount (in grams) of administrated T-lymphocytes. However, such a host versus graft reaction may also turn out to improve treatment efficiency, because of a strengthened inflammatory response within the tumour (second-set rejection).

Preferably, tumour cells actively killed by the T-lymphocyte cell lines of the present invention release endogenous adjuvants upon cell killing. Such adjuvants stimulate the patients own immune response (Shi et al., 2000). This implicates that the killed tumour cells actively participates in alarming the immune system that the tumour represents a danger. Hence, it is preferred that both the T-lymphocytes of the present invention and the tumour cells to be killed are important players in the attempt to activate the immune system by generating the necessary danger signals.

Soluble tumour associated peptide HLA complexes are released during tumour cell killing and such complexes could interfere with the interaction between T-lymphocytes and tumour cells. It is contained within the present invention if required, to remove such complexes from the blood stream during treatment. This can be done using any immuno-separation technique known to the person skilled in the art. By way of example this could be an immuno-magnetic separation technique or a separation technique comprising antibodies specifically binding said HLA complexes coupled to a solid material, which could for example be a column or beads. The presence of soluble melanoma peptide HLA complexes can serve as a marker for the effectiveness of tumour eradication.

A similar approach is applicable for other continuous T-lymphocyte cell lines. In particular it is applicable for CD4+ T lymphocyte cell lines. CD4+ T cell lines are in general HLA class II restricted. Such T cell lines can be chosen to have a type 2 cytokine profile (primarily characterised by a high ratio of IL-4/IFN-γ production) that may aid T-lymphocytes having a type I cytokine profile with a high IFN-γ/IL-4 ratio to activate the immune system of the patient.

Adoptive Immunotherapy Against Viral Diseases

It is another object of this invention to provide a method of prophylactic or therapeutic treatment of viral diseases, such as infection with HIV, CMV, EBV, HTLVI or HTLVII.

In one embodiment of the present invention continuous, normal T-lymphocyte cell lines expressing specific immune receptors that recognise HIV specific antigens are used in allogeneic adoptive immunotherapy against AIDS.

In another embodiment of the present invention continuous, normal T-lymphocyte cell lines expressing specific immune receptors that recognise cytomegalovirus (CMV) specific antigens are used in allogeneic adoptive immunotherapy against CMV infection. In particular, such treatment can restore deficient immunity to cytomegalovirus in allogeneic bone marrow transplant recipients. The bone marrow transplant recipients could be deficient in CMV-specific immunity due to ablation of host T cell responses by pretransplant chemotherapy, radiation therapy or a combination thereof.

T-Lymphocyte Cell Lines

The T-lymphocyte cell lines according to the present invention are preferrably derived from a human being, i.e. the cell lines are preferably human T-lymphocyte cell lines.

The T-lymphocyte cell lines to be used in this invention, can be derived from a tissue sample comprising disease activated cells, which sample is taken from a mammal including a human being. Alternatively, the T-lymphocytes can be derived by obtaining T-lymphocytes and antigen presenting cells (APCs) from a mammal including a human being, and activating such T-lymphocytes by e.g. mixing them with an antigen(s).

The T-lymphocyte lines may originate from a mammal being inflicted with a cancer or from a healthy mammal. Preferably, the tissue sample is a biopsy taken at the site of the disease. Such tissue sample is expected to further comprise antigen presenting cells as well as the antigen(s) that caused the activation of the T-lymphocytes.

The T-lymphocytes cell lines of the present invention are preferably derived from a tissue sample. The tissue sample is preferably selected from a biopsy, from sputum, swaps, gastric lavage, bronchial lavage, and intestinal lavage, or any body fluid such as spinal, pleural, pericardial, synovial, blood and bone marrow or from the spleen, the lymph nodes and thymus. More preferably said T-lymphocyte cell line is derived from a skin biopsy.

A biopsy can in principle be taken from any organ including the pancreas, the intestines, the liver, the kidneys, the lymph nodes, the breasts, and from the skin. Preferably the cells are taken from the organ of the disease.

In a preferred embodiment of this invention the T-lymphocyte lines are derived from patients with cutaneous T cell lymphoma, for example Sezary's syndrome. Most preferred the T-lymphocyte lines are derived from skin biopsies from patients with Sezary's syndrome.

In one embodiment of the present method, the disease associated T-lymphocytes are CD4+, CD8+ or CD4−/CD8− T-lymphocytes.

In particular, the disease associated T-lymphocytes are inflammatory, cytotoxic or regulatory T-lymphocytes.

T-lymphocytes of the present invention are preferably CD4+(positive), CD8+, or CD4−(negative)/CD8− T-lymphocytes. In one embodiment, regulatory T-lymphocytes are cytotoxic T-lymphocytes, or CD4+ T-lymphocytes, which in the case of a type 1 inflammation produce IL-4 or IL-10 and TGFβ, or in the case of a type 2 inflammation produce IFN-γ or IL-10 and TGFβ. In another embodiment, inflammatory T-lymphocytes are T-lymphocytes involved in chronic inflammatory/auto-immune diseases falling within the two major groups: A type 1 chronic inflammation dominated by production of primarily IFN-γ and TNFα (a type 1 inflammatory cytokine profile) or a type 2 chronic inflammation dominated by production of primarily IL-4 and IL-5 (a type 2 cytokine production).

In particular the T-lymphocytes of the present invention could originate from cytotoxic T-lymphocytes. In particular, such cytotoxic T-lymphocytes may have a CD8+phenotype. The cytotoxic T-lymphocytes are further preferably tumour infiltrating lymphocytes (TIL) or cells having similar properties. The selection of such cells are accomplished by addition of e.g. one of more additional compounds selected from GM-CSF, caspase inhibitors such as Z-VAD, α-CD95, IL-10, IL-12, IL-16, IL-18, IL-21, IFN-γ and functionally similar compounds or by any other conventional protocol.

In a preferred embodiment of this invention, the T-lymphocytes are cytoxic T-lymphocytes capable of tumour cell killing activity. In particular the T-lymphocytes are capable of killing tumour cells expressing an antigen recognised by the specific T-cell receptor expressed by said T-lymphocytes. In particular the T-lymphocytes are capable of killing tumour cells presenting an antigen in the context of a MHC molecule of the class recognised by the specific T-cell receptor expressed in said T-lymphocytes.

In a preferred embodiment the T-lymphocytes of this invention, when mixed with tumour cells presenting the specific antigen recognised by the T-cell receptor expressed in said T-lymphocytes, in the context of a MHC molecule of the class recognised by said T-cell receptor, in a ratio of 25:1, are able to kill more than 30%, such as more than 50%, such as around 65% in 4 hours as determined by a standard ⁵¹Cr release assay.

The activated T-lymphocyte lines of this invention preferably secrete one or more than one cytokine. The cytokine(s) could be selected from, but is not limited to, one or more of: IFN-γ, IL-10, TNFα, IL-12, IL-2, IL-4, GM-CSF, IL-5, IL-21 and TGFβ.

In a preferred embodiment activated T-lymphocytes of this invention secretes IFN-γ. In another preferred embodiment activated T-lymphocytes of this invention secretes GM-CSF. In another preferred embodiment activated T-lymphocytes of this invention secretes IL-5. In another preferred embodiment T-lymphocytes of this invention secretes TNF-α following activation. More preferably the T-lymphocytes of this invention following activation secretes a combination of two cytokines selected from IFN-γ, GM-CSF, IL-5 and GM-CSF. Even more preferably the T-lymphocytes of this invention following activation secretes a combination of three cytokines selected from IFN-γ, GM-CSF, IL-5 and TNF-α. Most preferably the T-lymphocytes of this invention following activation secretes a combination of four cytokines selected from IFN-γ, GM-CSF, IL-5 and TNF-α.

Preferably the activated T-lymphocyte lines of this invention secrete between 0,5 and 10 ng/ml/10⁶ cells/20 hours IL-5, more preferably between 1 and 8 ng/ml/10⁶ cells/20 hours IL-5, even more preferably between 2 and 6 ng/ml/10⁶ cells/20 hours IL-5, most preferably around 4 ng/ml/10⁶ cells/20 hours IL-5.

Preferably the activated T-lymphocyte lines of this invention secrete between 5 and 50 ng/ml/10⁶ cells/20 hours GM-CSF, more preferably between 10 and 40 ng/ml/10⁶ cells/20 hours GM-CSF, even more preferably between 20 and 30 ng/ml/10⁶ cells/20 hours GM-CSF, most preferably around 24 ng/ml/10⁶ cells/20 hours GM-CSF.

Preferably the activated T-lymphocyte lines of this invention secrete secretes between 0,5 and 10 ng/ml 10⁶ cells/20 hours IFN-γ, preferably between 1 and 8 ng/ml/10⁶ cells/20 hours IFN-γ, more preferably between 2 and 6 ng/ml/10⁶ cells/20 hours IFN-γ, most preferably around 4,5 ng/ml 10⁶ cells/20 hours IFN-γ.

Preferably the activated T-lymphocyte lines of this invention secrete between 0.5 and 10 ng/ml/10⁶ cells/20 hours TNF-α, more preferably between 1 and 8 ng/ml/10⁶ cells/20 hours TNF-α, even more preferably between 2 and 6 ng/ml/10⁶ cells/20 hours TNF-α, most preferably at least 1.5 ng/m/10⁶ cells/20 hours TNFα.

Cultivating Human, Continuous T Cell Lines

According to conventional state of the art it has been considered impossible to expand specific, human T-cell clones in sufficient numbers for immunotherapeutic trials, as T lymphocytes like other normal human somatic cells are believed to have a finite life span in vitro. (The definition here of a normal cell is here a cell of non-malignant origin). This limit of cell division is known as the “Hayflick limit” or replicative senescence.

Replicative senescence is measured by the number of cell population doublings (PD) cells in culture can expand to before cell proliferation definitive cease. Cell lines constrained by replicative senescence are known as finite cell lines. In the science of mammalian cell biology, it is a dogma that replicative senescence is an inevitable biological process that cannot be overcome by improved cell culture procedures. For human T lymphocytes the best estimate of replicative senescence is 23 PD (7). This means that one T cell on average can generate 2²³=10⁷ (approximately 10 milligram) T lymphocytes.

The present invention relates to growing at least 10⁹, such as at least 10¹⁰, for example at least 10¹², such as at least 10¹⁵, for example at least 10²⁰, such as at least 10³⁰, for example at least 10⁵ such as an in principle unlimited number of T-lymphocytes comprising nucleic acids encoding an immune receptor with defined specificity. This invention further relates to the use of said T-lymphocytes in immunotherapy, preferably allogeneic immunotherapy.

An approach for generating continuous T-lymphocyte lines from normal human T-lymphocytes, that are not constrained by replicative senescence i.e. they can undergo at least 30 PD, is described by the inventor in the PCT application WO 00/00587, which is hereby incorporated by reference. Cell lines with an apparent unlimited division capacity are known as continuous cell lines. The novel biological recognition that human T lymphocytes occasionally, but reproducibly can escape replicative senescence was observed when in vivo activated T lymphocytes were cultured in the presence of two T cell growth factors IL-2 and IL-4 (Kaltoft et al., 1992, Kaltoft et al., 1994, Kaltoft et al., 1995a, Kaltoft et al., 1995b).

Such continuous T cell lines are apparently activated in vivo in such a way that continuous growth can be obtained in a medium supplemented only with IL-2 and IL-4 but without addition of antigen and antigen presenting cells. It has furthermore been shown that continuous T cell lines during long term culture in the presence of high concentrations (more than 1 nM) of IL-2 and IL-4 maintain normal T-lymphocyte functions (Kaltoft et al., 1998, WO 00/00582).

Contrary to finite cell lines, continuous cell lines can generate an unlimited amount of T cells. All continuous T cell lines have progressed beyond 150 PD. By way of example it should be noted that an increase in PD from 23 (corresponding to approximately 10 mg. of cells) to “only” 50 PD will instead generate a cell mass of 2⁵⁰ cells=10¹⁵ cells equivalent to 1 ton of cells.

The inventor has established several continuous cytotoxic T cell lines. It has been shown (see example 1) that during long term culture these continuous T cell lines still have the ability to produce cytokine/chemokines and to act as killer cell (cytotoxic T cells, CTL). In a preferred embodiment of this invention, said cytotoxic, continuous T-lymphocyte lines are employed.

In accordance with the present invention, T-lymphocyte cell lines are preferably cultured in the presence of at least two factors which promote T-lymphocyte growth and/or maintains the phenotypical and functional integrity of continuous T-lymphocyte cell lines, and optionally one or more additional compounds which preferably are such as to directly or indirectly interfere with T-lymphocyte growth, in particular such which enhance or inhibit growth of inflammatory, regulatory or cytotoxic T-lymphocytes.

Factors which promote T-lymphocyte growth may be selected from the group consisting of cytokines which promote T-lymphocyte growth. Examples of such cytokines are IL-2, IL-15, IL-4, IL-7, IL-9, IL-10, IL-16, IL-21 and functionally similar cytokines. In particular, a combination of (1) IL-2 and/or IL-15, and (2) IL-4 and/or IL-7 and/or IL-9 may be used. In one embodiment of the present method, a combination of IL-2 and IL-4 is used. However, other T-lymphocyte growth promoting factors may also be used. Examples are combinations of ligation of the surface markers CD2, CD3 or CD28 with antibodies directed against CD2, CD3 or CD28.

By the term “functionally similar” is meant that the effect observed are comparable to the effect observed by the cytokines mentioned in the context of the present invention. These functionally similar compounds may substitute the specifically mentioned compounds in the specific process referred to.

The function of the additional compound is to promote the selection and expansion of a desired function of the T-lymphocytes (i.e. inflammatory or regulatory). When such additional compound or compounds is used, it may preferably be selected from cyclosporin, GM-CSF, Prednisone, Tacrolimus, FK506, IL-10, IL-10 antibody, TNFα antibody, IL-12, anti-IL-12, IL-7, anti-1L-7, IL-9, anti-IL-9, IL-16, caspase inhibitors, and similar compounds.

The cytokines are preferably used in a concentration of at least 1 nM each, preferably more than 2.5 nM, more preferably than 10 nM each. The concentration of the cytokines might not be important, however, the concentration should be chosen so as to ensure growth and normal T-lymphocyte function, i.e. at least 1 nM of each. Traditionally, the concentration of a cytokine is expressed as activity in units per ml (u/ml). The person skilled in the art will readily know how to interrelate u/ml and concentration (molar, M). If nothing else is stated, it is to be assumed that 200 u/ml equals 1 nM.

In one preferred embodiment the T-lymphocytes cell lines are cultivated in the presence of at least 1 nM IL-2, such as at least 1.5 nM IL-2, for example at least 2.0 nM IL-2, such as at least 2.5 nM IL-2, for example at least 3 nM IL-2, such as 3.5 nM IL-2, for example at least 4 nM IL-2, such as 4.5 nM IL-2, for example at least 5 nM IL-2, such as 5.5 nM IL-2, for example at least 6 nM IL-2, such as 6.5 nM IL-2, for example at least 7 nM IL-2, such as 7.5 nM IL-2, for example at least 8 nM IL-2, such 8.5 nM IL-2, for example at 9 nM IL-2, such as 9.5 nM IL-2, for example more than 10 nM IL-2.

In one preferred embodiment the T-lymphocytes cell lines are cultivated in the presence of at least 1 nM IL-4, such as at least 1.5 nM IL-4, for example at least 2.0 nM IL-4, such as at least 2.5 nM IL-4, for example at least 3 nM IL-4, such as 3.5 nM IL-4, for example at least 4 nM IL-4, such as 4.5 nM IL-4, for example at least 5 nM IL-4, such as 5.5 nM IL-4, for example at least 6 nM IL-4, such as 6.5 nM IL-4, for example at least 7 nM IL-4, such as 7.5 nM IL-4, for example at least 8 nM IL-4, such 8.5 nM IL-4, for example at 9 nM IL-4, such as 9.5 nM IL-4, for example more than 10 nM IL-4.

In one preferred embodiment IL-12 is added to the T-lymphocyte cell culture approximately one day prior to administration. Preferably, at least 10 pM IL-12 is added, more preferably at least 50 pM IL-12 is added, even more preferably between 50 and 150 pM IL-12 is added, most preferably around 100 pM IL-12 is added. Addition of IL-12 increases the production of IFN-γ by the T-lymphocytes of the present invention.

Apart from antigen activation, any other non-specific method that is available and promote T-lymphocyte growth can be applied in cases where the cell population doubling time is considered too long. Such methods include activation by super-antigen pulsed antigen presenting cells, activation by mitogens (like PHA and jacalin) in the presence of feeder cells or antigen presenting cells, activation by antibodies against CD2, CD3 and CD28, activation by ionomycin and phorbol ester and in case of cross-reactivity with alloantigen, allostimulation with appropriate allogeneic cells with or without autologous dendritic cells (the latter possibility in order to obtain cross-priming). AICD can in all the cases mentioned above be blocked by caspase inhibitors.

Expression of Antigen Specific Immune Receptors in Continuous T Cell Lines

The T-lymphocyte cell lines according to the present invention are preferably capable of expressing at least one antigen specific immune receptor encoded by at least one heterologous nucleic acid sequence. By the term heterologous nucleic encoding an antigen specific immune receptor is meant a nucleotide sequence encoding an antigen specific immune receptor operably linked to an expression signal not natively associated therewith.

The T-lymphocyte cell lines are preferably capable of expressing sufficient amounts of the antigen specific immune receptor in order to for the T-lymphocytes to recognise the specific antigen. By way of example, when the T-lymphocyte cell line is a cytotoxic T-lymphocyte cell line, then expression of the antigen specific immune receptor should be sufficient for the cytotoxic T-lymphocytes to obtain cytotoxic activity against cells expressing said specific antigen.

Hence, the expression of the antigen specific immune receptor should preferably be detectable by conventional techniques such as Western blotting or ELISA.

More preferably, the T-lymphocyte cell lines are capable of continuously expressing the at least one antigen specific immune receptor. “Continously expressing” is used herein interchangeable with “stably expressing” and the terms are meant to cover that the transgenic antigen specific immune receptor is expressed at a stable level regardless of the number of population doublings, that the T-lymphocyte cell lines has undergone since introduction of the nucleic acid sequence encoding said antigen specific immune receptor.

Hence, preferably the expression level of the antigen specific immune receptor of a T-lymphocyte cell line according to the present invention is at least 30%, such as at least 40%, for example at least 50%, such as at least 60%, for example at least 70%, such as at least 80%, for example at least 90%, such as at least 95%, for example at least 97%, such as at least 99%, for example essentially 100% of the initial expression level of said antigen specific immune receptor after 10, such as after 20, for example after 30, such as after 40, for example after 50, such as after 60, for example after 70, such as after 80, for example after 90, such as after 100, for example after 150, such as after 200, for example after 250, such as after 300, for example after 500 population doublings.

The “initial expression level” is the level of expression of said antigen specific immune receptor obtained 24 hours, such as 48 hours, for example 3 days, such as 4 days, for example 5, days, such as 6 days, for example one week after introduction of the nucleic acid encoding said antigen specific immune receptor. In addition it is preferred that the at least one antigen specific immune receptor encoded by at least one heterologous nucleic acid sequence is capable of inducing appropriate signal transduction in T-lymphocytes expressing said antigen specific immune receptor.

In a preferred embodiment of the present invention the T-lymphocytes cell lines are capable of stably expressing an antigen specific immune receptor encoded by at least one heterologous nucleic acid sequence, wherein said antigen specific immune receptor inducing reactivity of said T-lymphocyte cell line upon association with the specific antigen.

The nature of said induced reactivity of the T-lymphocytes cell line is dependent on the specific T-lymphocyte cell line. By way of example, if the T-lymphocyte cell line is a cytotoxic T-lymphocyte cell line, then reactivity may include cytotoxic activity and/or cytokine production.

Cytotoxic activity is preferably cytotoxic activity against cells expressing the specific antigen. The cytotoxic activity may for example be determined by a standard ⁵¹Cr release assay.

Cytokine production may for example include production of one or more cytokines selected from the group consisting of TNF, GM-CSF, IFN-β, IL-5 and IL-8. Cytokine production may be determined by any conventional assay known to the person skilled in the art, for example by an ELISA assay.

It is preferred that appropriate signal transduction is induced in T-lymphocyte cell lines expressing the antigen specific immune receptor, when said T-lymphocyte cell lines encounter the specific antigen. Accordingly it is preferred that the antigen specific immune receptor is capable of associating with the specific antigen after expression in T-lymphocytes.

“Appropriate signal tranduction” depends on the specific T-lymphocyte and the specific antigen specific immune receptor. In one embodiment of the present invention “appropriate signal transduction” may be determined by achievement of one or more desired outcome of said signal transduction. For example “appropriate signal transduction” may i.a. result in production of one or more components selected from the group consisting of TNF, GM-CSF, IFN-γ, IL-5 and IL-8.

Furthermore, “appropriate signal transduction” may for example result in cytotoxic activity. It is usually preferred that “appropriate signal transduction” results in at least increased production of TNF.

By way of example, if the antigen specific immune receptor is a T-cell receptor recognising Mart-1, then preferably upon recognition of Mart-1 by a T-lymphocyte expressing said T-cell receptor, then appropriate signal tranduction is induced resulting in for example TNF production and cytotoxic activity against cells expressing Mart-1.

It is possible that the T-lymphocyte cell line apart from expressing antigen specific immune receptor(s) encoded by heterologous nucleic acid sequences (herein after “transgenic antigen specific immune receptors), also express one or more antigen specific immune receptor, which is encoded by nucleic acid sequences comprised within the genome of said T-lymphocyte cell lines. Such antigen specific immune receptors are referred to as “endogenous immune receptors” herein after.

A T-lymphocyte may thus express both transgenic antigen specific immune receptors and endogenous immune receptors. Hence it is preferred that at least the transgenic antigen specific immune receptors are capable of inducing appropriate signal transduction.

In specific embodiments of the present invention the T-lymphocyte cell lines do express a reduced amount of functional endogenous immune receptors, i.e. the T-lymphocyte cell lines may for example not express detectable endogenous T-cell receptor. Hence it is preferred that the T-lymphocyte cell lines for example do not express the α-chain and/or the β-chain of endogenous T-cell receptors.

The continuous T-Lymphocyte line C-Cure 709 and safety of clinical use thereof.

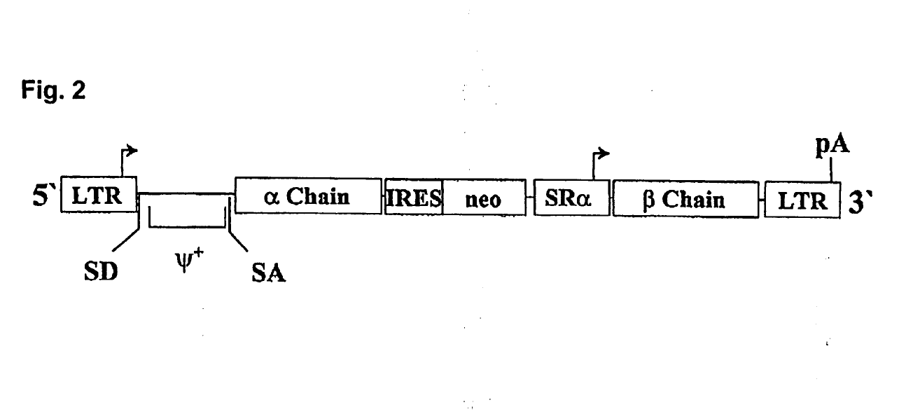

In one preferred embodiment, the present invention relates to a continuous T-lymphocyte cell line in the form of C-Cure 709 as deposited with ECACC accession number 01030609. C-Cure 709 is a continuous T-lymphocyte cell line expressing a T-cell receptor specific for the M9-2 peptide of MART-1, from the A7 construct (see FIG. 2). It is derived from C-Cure 707 as deposited with ECACC acccesion number 01030608.

The immune system does not in general consider cancer cells to be dangerous, although cancer is clearly life threatening for the individual. If immunotherapy is to succeed the immune system has to realise that the cancer represents a danger.

In one preferred embodiment the present invention relates to the inflammatory nature of the continuous T-cell line C-Cure 709. C-Cure 709 has the potential to alarm and activate the immune system. An important feature of C-Cure 709 is its ability both directly and indirectly to activate cells of the immune system. By direct injection into the edge of the tumor tissue the following scenario may at best occur

(see FIG. 1)

C-Cure 709 recognises HLA-A2+ melanoma cells expressing Mart-1 by melanoma cell killing. Melanoma cells actively killed by C-Cure 709 releases by cell killing endogenous adjuvants that stimulate the patients own immune response (Shi et al., 2000). Upon killing melanoma cells C-Cure 709 releases cytokines like TNF-α (table 2). TNF-α may by itself exert tumor cell killing (a so-called bystander effect).

Experiments have shown that C-Cure 709 is not capable of transferring the A7 construct to other continues T cell lines like C-Cure 703 or C-Cure 707. This agrees with data showing that the A7 construct is a non-replication competent viral construct. The risk of transfer of the A7 vector to hospital personal is therefore considered extremely low. As human serum lyses retroviral particles from the PG13 package cell line this is expected to further minimise the risk of transfer of the A7 transgene.

As a treatment modality C-Cure 709 is an allogeneic genetically modified continuous γ-irradiated T cell line. Injection in to a tumour site corresponds in principle to a transplantation or transfusion: C-Cure 709 is a HLA mismatched donor (graft) and the patient is the host. A host versus graft reaction against C-Cure 709 may thus be expected after 10-14 days after the first injection (a first-set rejection).

As for (allogeneic) transplantation in general this reaction will most likely primarily be directed against HLA class I and II antigens on C-Cure 709. Besides these immunodominant molecules the transgene A7 T cell receptor and the neomycin phosphotransferase gene are immunogenic, as they are neo-antigens. Other allogeneic differences between donor and graft can of course also give rise to a host versus graft reaction. These immune reactions are not expected to provoke severe side effects, because of the relative low amount (in grams) of injected C-Cure 709 suggested in the treatment protocol. It is considered that the worst outcome of repeated injections of C-Cure 709 is that a possible treatment effect will end after approximately 14 days due to a host versus graft reaction.

However, such a host versus graft reaction may also turn out to improve treatment efficiency, because of a strengthened inflammatory response within the tumour (second-set rejection). In case a host versus graft reaction leads to reduced efficacy of C-Cure 709, another non HLA matched continuous melanoma specific T cell line could ideally substitute for C-Cure 709.

In previous clinical trials allogeneic melanoma cell lines, chemically or genetically modified allogeneic melanoma cell lines (expressing cytokine genes or co-stimulatory molecules) have shown no severe side effects.

The main risk of the suggested C-Cure 709 protocol is that the inflammation generated by this inflammatory T cell will start an unwanted autoimmune process initiated by autologous antigen presenting cells such as dendritic cells. In a paper published in Nature Medicine (Nestle et al., 1998) 16 patients with malignant melanoma were repeatedly (up to 10 times) vaccinated with autologous tumour peptide pulsed dendritic cells (10⁶/vaccination). In this phase I trial there was no physical evidence of autoimmune disease. In two other relevant clinical trials (Kugler et al., 2000, Trefzer et al., 2000) patients with renal cell carcinoma (17 patients) and malignant melanoma (16 patients) were treated with allogeneic dendritic cells (5 10⁷) or B-cells fused with autologous tumour cells These studies showed no evidence of autoimmune disease expect for vitiligo in the case of malignant melanoma. In this context it should here be noted that allogeneic dendritic cells are capable of directly activating the patients own alloreactive T lymphocytes. As the amount of allogeneic dendritic cells in these studies were 510⁷, the initial amount of C-Cure 709 cells injected to a patient with malignant melanoma is suggested to be the same. This amount is far less than what has been used in the TIL protocols. An allogeneic γ-irradiated leukemic cell line TALL-104 has in a phase I trial been given intravenously to 15 patients with metastatic mamma cancer. Up to 10⁸ cells/kg were transferred, resulting in one case of grade IV toxicity, the remaining patients only experienced mild grade I/II toxicity (Visonneau et al., 2000)

The quality control of C-Cure 709 is performed weekly and consists of a careful monitoring of phenotype and function. These investigations are supplemented with HLA typing together with examination of normal variations in heteromorphic regions of Q-banded chromosomes. These investigations will assure that C-Cure 709 is authentic and that there is no sign of cross contamination with other cell lines.

C-Cure 709 will 3 days before injection be cultured in a medium with serum from the patient. The serum and the used culture medium will be serologically tested for HIV status, hepatitis ABC, HTLV-1, CMV, EBV, antinuclear antibodies (ANA), and anti neutrophil cytoplasmatic antibodies (ANCA). Test for the absence of mycoplasma will also be performed.

Antigen Specific Immune Receptors

It is possible by means of recombinant DNA technology to generate vectors that encode specific and functional immune receptors. The present invention demonstrates that it is feasible to transfer tumour specific immune receptors into continuous T cell lines and thereby redirect their specificity to tumour cell recognition.

One example of a recombinant melanoma specific T cell receptor that can be used in combination with continuous T cell lines to treat a large fraction of malignant melanoma patients is the A7 T cell receptor (see FIG. 2). In example 1 of the present invention, the specificity of a CD8+ continuous clonal T cell line (C-Cure 707) is redirected by introduction of nuleic acid sequences encoding the A7 T cell receptor (FIG. 2).

It is within the scope of the present invention to generate several other recombinant T cell receptors, which could be both HLA class I and HLA class II restricted. This would eventually lead to a library of tumour specific T lymphocytes cell lines as disclosed in the present invention, covering the whole spectrum of tumour patients. A similar approach for specific T cell receptors recognising various disease specific antigens could broaden the application to other clinical conditions such as for example viral diseases.

In one embodiment of present invention the specificity of continuous T cell lines is redirected by introduction of nucleic acid sequences encoding one or more antigen specific immune receptors into said T-lymphocytes. Preferably, the antigen specific immune receptors are selected from the group consisting of: T-cell receptors and chimeric immune receptors.

Chimeric immune-receptors consist in general of a tumour/virus specific antibody binding part (single chain Fragment variable, scFv) coupled to a T lymphocyte signalling unit. A T lymphocyte signalling unit could for example be the ξ chain of CD3. The advantage of using chimeric immune receptors is that antibody specificity and avidity against antigens is in general better than that of TCR's. A further advantage is that chimeric immune receptors are not HLA restricted. In addition antibodies can often directly monitor the expression of chimeric receptors in continuous T cell lines. This is for instance still not possible for the A7 T cell receptor because no antibodies exist that specifically detect the subfamilies of the A7 T cell receptor (α1.1 and β73). The detection of the expression of the A7 receptor relies as shown mostly on functional assays.

Examples of chimeric immune receptors (reviewed in Abken et al., 1998) are chimeric receptors recognising: the tumour antigen TAG-72 present on most adenocarcinomas; HER/neu expressed on some breast, gastric, colon and ovarian carcinomas; CA724 expressed on carcinomas; ovarian adenocarcinomas expressing the 38 kDa folate-binding protein; renal carcinoma expressing the G250 protein; gastrointestinal carcinoma expressing carcinoembryonic antigen, Hodgkin's lymphoma expressing CD30 and melanoma expressing the high-molecular-weight melanoma-associated antigen (HMW-MAA); and tumours expressing the CD44v6 splice variant. The HMW-MM antibody scFv has the designation 763.74 and a Fab fragment coupled to modified superantigens is called K305 (see example 2).

Tumour Specific T-Cell Receptors

In one embodiment of the present invention continuous T-lymphocyte cell lines comprising nucleic acid sequences encoding a specific T-cell receptor as described in WO 96/30516, which is hereby incorporated in its entirety, are described.

Accordingly, in a preferred embodiment this invention relates to continuous T-lymphocyte cell lines which comprise exogenous T-cell receptors which recognise or bind tumour associated antigens presented in the context of MHC Class I. In another preferred embodiment the tumour associated antigens recognised by the T-cell receptors of this invention are melanoma antigens.

By way of example the melanoma specific T-cell receptors of this invention may recognise melanoma antigens in the context of HLA-A2.1 or HLA-A1. Examples of melanoma antigens that are recognised by the T-cell receptors include, but are not limited to, MART-1. In a preferred embodiment the T-cell receptor recognises or binds to the MART-1 peptide, in particular epitopes M9-1 (TTAEEMGI), M9-2 (MGIGILTV), M10-3 (EMGIGILTV), and M10-4 (AAGIGILTVI) (shown in single letter amino acid code) or gp-100 peptide epitopes.

The functional α-chain of the heterodimeric T-cell receptors of this invention may have the following formula:

V-J-C

wherein,

V is an amino acid sequence comprising the variable region of the α-chain. By way of example, the V gene after rearrangement may have a 3′ end encoding for a carboxy terminus sequence of Cysteine-Xaaₙ where n may be about 1-5 and Xaa may be any amino acid or a combination of amino acids. Preferably Xaa is Alanine or Serine. In a preferred embodiment, the 3′ end of the V gene encodes for a carboxy terminus of Cysteine-Alanine. Examples of V α-genes that be may be used in generating this region include, but are not limited to, Vα8.2 or Vα17, Vα9, Vα1, Vα25, or Vα21.

J denotes the joining region. Examples of J genes that may be used to generate this region, include but are not limited to, Jα49, Jα42, Jα16, or Jα54. C denotes the constant region of the α-chain.

The functional P chain of the heterodimer T-cell receptors may have the formula:

V-D-J-C

wherein

V is an amino acid sequence comprising the variable region of the β chain. The V gene may have a 3′ end encoding for a carboxy terminus of Cysteine-Xaaₙ wherein n may be about 1-5 and Xaa may be any amino acid or combination of amino acids. Preferably, Xaa is either Alanine or Serine. In a preferred embodiment, the 3′ end of the V region encodes for a carboxy terminus of Cysteine-Alanine-Serine, or Cysteine-Alanine-Serine-Serine, or Cysteine-Alanine. Examples of V genes that may be used for the V region include but are not limited to Vβ13.6, Vβ6.5, Vβ22.1, Vβ7.3, or Vβ3.1.

J denotes the joining region. Examples of Jβ genes that may be used in generating the joining regions include, but are not limited to, Jβ1.5, Jβ2.1, Jβ1.1, or Jβ2.7.

Examples of D (diversity) genes that may be used include, but are not limited to Dβ1.1, or Dβ2.1.

C denotes the constant regions of the β chain. Examples of constant regions that may be used, include, but are not limited to Cβ1 in Cβ2.

In one embodiment the T-cell receptor of this invention comprises a nucleic acid sequence encoding for a variable region having a 3′ encoding for a carboxy terminus of Cysteine-Xaaₙ, a J region and a constant region in combination with a β chain comprising a nucleic acid sequence encoding for a variable region having a 3′ end encoding for carboxy terminus of Cysteine Xaaₙ, a D region and a J region and a constant region. The alpha and beta chains of the T-cell receptors form a ligand binding domain that preferably recognises a tumour associated antigen, most preferably melanoma antigens.

In the preferred embodiments the melanoma specific T-cell receptors provided herein have the following α and β chain combinations: Vα8.21J 49/C chain and Vβ13.6/Dβ1.1/Jβ1.5/Cα1; Vα17/Jα42/Cαand Vβ6.5/Dβ1.1/Jβ1.5/C1; Vα9/Jα16/Cα and Vβ22.1/Dβ2.1/Jβ2.1/Cβ2; Val/Jα49/Cα and Vβ7.3/Dβ2.1/Jβ2.1/Cβ2; Vα25/Jα54/Cα and Vβ3.1/Dβ1.1/Jβ1.1/Cβ; Vα2/Jα42/Cα and Vβ7.3/Dβ2.1β/Jβ2.7/Cβ2

Tumour Associated Antigens

The antigens recognised by the T-cell receptors of this invention are preferably one or more antigens specific for the cancer to be treated. The cancer could be selected from any of the above mentioned.

In particular, the antigens could be melanoma specific antigens. The melanoma specific antigen could be a peptide derived from a melanoma specific protein selected from the following: tyrosinase, MART-1 and/or gp100. The antigen could be presented in context with any MHC molecule and/or alone.

By way of example, if a patient expresses HLA-A2 (HLA 0201), the immunogenic melanoma associated peptides restricted by this HLA allele are known to derive from at least the following proteins: Tyrosinase, Melan-A/Mart-1 and gp100. The amino acid sequence of the HLA-A2 binding melanoma associated peptides is for tyrosinase MLLAVLYCL, for Melan-A/Mart-1 MGIGILTV (M9-2), and for gp100 KTWGQYWQV.

HLA-A2 is an allele, which more than 50% of Caucasians carry. Patients with metastatic malignant melanoma have a very poor prognosis with a median survival time of only 7.5 months. Accordingly it is desirable to have access to treatment options that can work fast. In one embodiment the present invention provides pre-made continuous T-lymphocyte cell lines, comprising HLA restricted recombinant T-cell receptors, for example HLA-A2 restricted T-cell receptors. As the vast majority of HLA-matched melanoma patients express the same tumour associated antigens, it is possible to establish T-lymphocyte cell lines as described in this invention that could fit every patient with malignant melanoma.

MART-1

Malignant melanoma is considered as one of the most immunogenic tumours. In particular, certain amino acid sequences of the protein Mart-1 that are expressed only in melanoma cells and melanocytes are known to be very immunogenic (14).

Mart-1 is a transmembrane protein of still unknown function. Mart-1 is like tyrosinase a differentiation antigen that belongs to a group of proteins that are expressed by both the normal pigment cell (the melanocyte) and the malignant pigment cell (the melanoma cell). Recognition of the immune system of Mart-1 can be expected to initiate an autoimmune process leading to cell destruction, which in the case of melanoma is desirable.

Melanocytes will possibly also be destroyed leading to a condition known as vitiligo. Vitiligo is characterised by paleness of local skin areas due to the destruction of the pigment cells. This response has in previous studies been positively correlated with the efficiency of IL-2 based immunotherapy of malignant melanoma (Rosenberg et al., 1996).

Expression Vectors

The nucleotide sequences encoding a specific immune receptor including a T cell receptor should preferably be comprised within one or more expression vector(s) and operably linked to expression control sequences suitable for expression in mammalian T-lymphocytes.

A vector is a replicable construct which could be any nucleic acid Including DNA, RNA, LNA and PNA. Once transformed into a suitable host, the vector replicates and functions either independently of the host genome or integrate into the genome itself. Any vector capable of replicating in a T-lymphocyte can be used.

The vector could be a viral derived vector, a retroviral derived vector, a phage, a plasmid, a cosmid, an integratable DNA fragment (i.e., integratable into the host genome by recombination), bacteria or eukaryotic cells.

In one preferred embodiment the expression construct(s) of the present invention comprises a viral based vector, such as a DNA viral based vector, an RNA viral based vector, or a chimeric viral based vector.

Examples of DNA viruses are cytomegalo virus, Herpex Simplex, Epstein-Barr virus, Simian virus 40, Bovine papillomavirus, Adeno-associated virus, Adenovirus, Vaccinia virus, and Baculo virus. Examples of RNA virus are Semliki Forest virus, Sindbis virus, Poko virus, Rabies virus, Influenza virus, SV5, Respiratory Syncytial virus, Venezuela equine encephalitis virus, Kunjin virus, Sendai virus, Vesicular stomatitisvirus, lentivirus and Retroviruses.

DNA regions are operably linked when they are functionally related to each other. For example, a promoter is operably linked to a coding sequence if it controls the transcription of the sequence; or a ribosome binding site is operably linked to a coding sequence if it is positioned so as to permit translation. Generally, operably linked means contiguous and, in the case of secretory leaders, contiguous and in reading phase.

Expression control sequences suitable for use herein may be derived from a prokaryotic source, an eukaryotic source including a mammalian source, a virus or viral vector or from a linear or circular plasmid. Further, the regulatory sequence can be a synthetic sequence, for example, one made by combining the UAS of one gene with the remainder of a requisite promoter from another gene.

The promoter regions are selected to be different from the native T-cell receptor promoters, and preferably, the promoter region is selected to function most optimally with the employed vector in T-lymphocytes. Commonly used promoters are derived from polyoma, Adenovirus 2 or Simian Virus 40 (SV40). Further, it is also possible, and often desirable, to utilise a mammalian genomic promoter. Any expression signal capable of directing gene expression in a T-lymphocyte is preferred in accordance with the present invention.

The promoter could be tissue-specific i.e. a transcriptional promoter/enhancer or locus defining elements, or other elements which control gene expression as discussed above, which are preferentially active in T-lymphocytes.

The promoter could be event-specific i.e. transcriptional promoter/enhancer or locus defining elements, or other elements which control gene expression as discussed above, whose transcriptional activity is altered upon response to cellular stimuli. Representative examples of such event-specific promoters include thymidine kinase or thymidilate synthase promoters, P interferon promoters, promoters responding to tetracyclin, promoters inducible by metal ions and promoters that respond to the presence of hormones (either natural, synthetic or from other non-host organisms, e.g., insect hormones).

Preferred promoter regions are Moline murine leukemia virus long terminal repeat and a hybrid HTLV-I/SV40 SRα promoter. The expression vector(s) should preferably also include a selectable marker. Suitable selectable markers in a mammalian host cell includes Neomycin, SV₂ Neo, TK, hygromycin, phleomycin, histidinol, or dihydrofolate reductase DHFR or any other suitbaly selectable marker.

The nucleotide sequences encoding the α and/or β chains of a specific T-cell receptor may be contained within the same expression vector or they could be contained within different expression vectors.

If contained within the same expression vector, the nucleotide sequences encoding the α or β chains of a specific T-cell receptor may be separated by an IRES (internal ribosomal entry site) and transcribed into one mRNA or they could be transcribed into separate mRNAs from different promoters.

Said nucleic acid sequence could be transferred to said T-lymphocytes by any method known to a person skilled in the art. Such method could be selected from, but is not restricted to electroporation, microinjection, lipofection with for example cationic liposomes, calcium phosphate precipitation, viral transfer, retroviral transfer, adsorption, bio-ballistic transfer by for example coated gold particles and protoplast fusion.

In a preferred embodiment of the present invention the nucleic acid sequence encoding said T-lymphocyte recptors is transferred to the T-lymphocytes by retroviral transfer.

Pharmaceutical Compositions

The present invention also relates to pharmaceutical compositions comprising a pharmaceutical effective amount of one or more continuous T-lymphocyte cell lines comprising an antigen specific immune receptor as described herein, optionally comprising one or more pharmaceutically acceptable drugs and/or excipients.

The continuous T-lymphocyte cell lines comprising an antigen specific immune receptor to be used in the composition are preferably inflammatory T-lymphocytes, regulatory T-lymphocytes, or cytotoxic T-lymphocytes. In one embodiment, the composition comprises one or more of said T-lymphocyte cell lines which have been activated in the presence of one or more antigens. Such antigens may preferably be tumour associated antigen(s), viral antigen(s), alloantigen(s), or super-antigen(s).

The T-lymphocytes are preferably attenuated prior to administration in order to ensure that the cells are not able to divide further. Such attenuation may suitably be accomplished by x-ray or UV radiation or by addition of cell poisons. More preferably the T-lymphocytes are lethally irradiated with γ-radiation prior to administration. In a preferred embodiment the T-lymphocytes are irradiated With 40-100 Gy γ-irradiation, more preferably 50-80 Gy, most preferably 60 Gy.

In order to reduce activation induced cell death (AICD), it is part of this invention prior to administration to incubate the T-lymphocytes with an inhibitor of AICD. Alternatively the inhibitor may be administrated together with the T-lymphocytes, either as a combination or sequentially in any order. Examples of such inhibitors are caspase inhibitors like Z-VAD and certain antibodies with reactivity to CD95 (Fas) that prevents Fas-FasL induced cell death.

The parenteral formulations can be presented in unit-dose or multi-dose sealed containers, such as ampoules, pre-filled syringes and vials, and can be stored in a freeze-dried (lyophilized) condition requiring only the addition of the sterile liquid excipient, for example, water, for injections, immediately prior to use. Preferably, the pharmaceutical composition of the present invention is a suspension in a physiological solution e.g. sterile isotonic, pyrogen-free water or serum. More preferably, the pahrmaceutical composition is a suspension in serum from the patient to be treated. Additionally the pharmaceutical composition may contain stabilisers, preservatives, PH-buffering agents, salts and the like.

The parenteral formulations typically will contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Administration

The suitable amount of the T-lymphocytes of the invention to be administered depends on several factors, i.a. the disease and the severity of the disease to be treated, alleviated or prevented. Further on the age, weight and state of the subject to be treated, the particular drug composition employed and on the route of administration. In general, 10⁵-10¹² cells may be suitable for each dose, preferably 10⁶-10¹⁰ cells per dose, more preferably 5×10⁷-5×10⁸ cell per dose.

Preferably the volume to be injected per unit dose range from 0,01 ml to 5 ml, more preferred from 0,1 ml to 2,5 ml, most preferred from 0,1 ml to 1 ml.

The administration may be as single doses or as several doses. In certain cases, administration only once may be sufficient. In general, several doses should be given such as once for a period of for examples a day, two days, three days, for a week or for months, or repeated administration once every second day, every third day, every week, every second week, etc. In another embodiment several doses is given with irregular intervals over a period of one week, two weeks, one month, two months, three month, six months, one year or longer.

In one embodiment the pharmaceutical composition is administrated following a 28 days schedule (table 6) such as a unit dose is given on day 1, 4, 7, 10, 14 and 28.

This scheme may be repeated once or more than once, especially, patients with no clinical progression after the first series of treatment can be offered a second identical treatment series.

TABLE 1
Suggested schedule for treatment of patients

| | | | | | | |
|---|---|---|---|---|---|---|
| | | | | | | Week 1 |
| Monday | Tuesday | Wednes- | Thursday | Friday | Saturday | Sonday |
| | Day 1 | day | | Day 4 | | |
| | | | | | | Week 2 |
| Monday | Tuesday | Wednes- | Thursday | Friday | Saturday | Sonday |
| Day 7 | | day | Day 10 | | | |
| | | | | | | Week 3 |
| Monday | Tuesday | Wednes- | Thursday | Friday | Saturday | Sonday |
| Day 14 | | day | | | | |
| | | | | | | Week 4 |
| Monday | Tuesday | Wednes- | Thursday | Friday | Saturday | Sonday |
| | | day | | | | Week 5 |
| Monday | Tuesday | Wednes- | Thursday | Friday | Saturday | Sonday |
| Day 28 | | day | | | | |
| | | | | | | Week 6 |
| Monday | Tuesday | Wednes- | Thursday | Friday | Saturday | Sonday |
| | | day | Evaluation | | | |

The pharmaceutical composition can be administrated parenterally for example by bolus injection or by continuous infusion either subcutaneously, intramuscularly, intravenously or intraperitoneally. More preferably the pharmaceutical composition is injected directly into the edge of a tumour, which could be the primary tumour or one or more metastatic lesions. It is preferred that the pharmaceutical composition is injected into at least one primary or metastatic location, more preferably the pharmaceutical composition is injected into at least two primary or metastatic locations.

### EXAMPLES

Deposition of Biological Material

The cell lines C-CURE 707 and C-CURE 709 have been deposited on 6 Mar. 2001 according to the Budapest Treaty with the “European Collection of Cell Cultures” (Salisbury, Wiltshire, SP4 OJG) with the below mentioned accession numbers.

| Cell name | Accession Number |
|---|---|
| C-CURE 707 | 01030608 |
| C-CURE 709 | 01030609 |

The following examples describe selected, preferred embodiments, and the invention should not be regarded as limited to the examples.

### Example 1

C-CURE 709, a Melanoma Specific T Cell Line

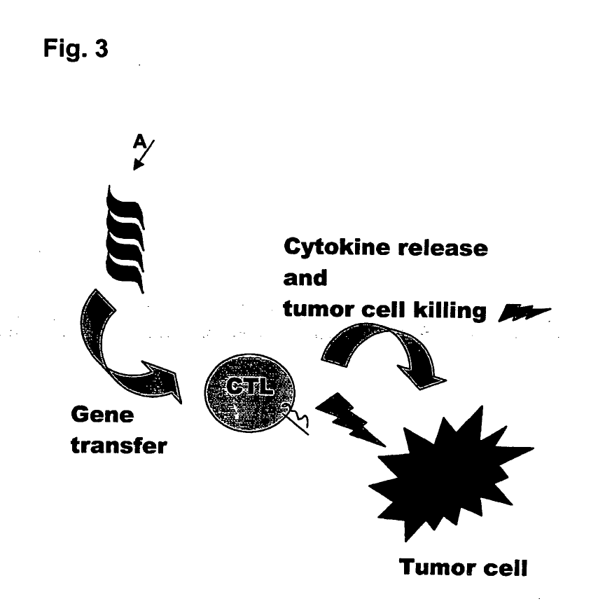

In order to test whether the above mentioned concept could be carried out in practice an attempt was made to transfer the A7 melanoma specific T cell receptor into continuous cytotoxic T cell lines. The model system is as illustrated in FIG. 3.

A7 T-Cell Receptor

We have from Dr. Michael Nishimura National Institute of Health, Bethesda, U.S.A. obtained the retroviral vector A7 (FIG. 2) encoding the complete T cell receptor recognising the Mart-1 M9-2 peptide restricted by HLA-A2 (Clay et al., 1999) (HLA-A2 belongs to HLA class I molecules). The A7 T cell receptor was cloned from a TIL cell culture of a melanoma specimen as previously described in detail (Clay et al., 1999). In short the important features of the retroviral A7 construct is given in the legend to FIG. 2. Dr. Michael Nishimura has informed that both the A7 T cell receptor construct as well as the retroviral package cell line PG13 that propagates the non-replication competent A7 retroviral construct has been approved for clinical trials by the American health authorities FDA.

Experiments performed in Dr. Nishimura's laboratory have shown that the A7 T cell receptor can be transferred to normal finite human T lymphocytes by supernatant from the A7/PG13 cell line. These transfected human T lymphocytes recognise melanoma cells expressing Mart-1 in the context of HLA-A2 (Clay et al., 1999). One such melanoma cell line is called 888-A2, which upon encounter with the transfected lymphocytes is killed often followed by cytokine production (Clay et al, 1999). The transfected T cell lines may have immunotherapeutical potential to treat the patient from whom they derive. However as this approach is individual, it is time consuming, laborious and costly, if many patients have to be treated on an individual basis. As explained above continuous cancer specific T cell lines do not have these drawbacks. Another major advantage of continuous cancer specific T cell lines is that they represent universal immunotherapeutic reagents that can be thoroughly tested like any other pharmaceutic agent.

C-Cure 701. 702. 703 and 704

In the first series of experiments four continuous clonal cytotoxic T cell lines C-Cure 701, C-Cure 702, C-Cure 703 and C-Cure 704 were transfected with the A7 T cell receptor employing the standard procedure described the for finite T lymphocyte cell lines (Clay et al., 1999). Selection for the A7 transgene transfer was done in the presence of the antibiotic G418 as the A7 construct confers resistance to this antibiotic by means of the neo gene (FIG. 2). All four cell lines could after (but not before) addition of the A7/PG13 supernatant be cultured in the presence of G418, indicating transfer of the A7 vector. However the transfected C-Cure 701 and C-Cure 704 showed no reactivity against the above mentioned 888-A2 melanoma cell line. The transfected C-Cure 702 and C-Cure 703 cell lines initially recognised 888-A2 melanoma cells as monitored by melanoma cell killing and cytokine production. Upon further expansion of these two cell lines the recognition of 888-A2 was gradually lost. It was in these experiments not possible to obtain a continuous T cell line with a stable and functional A7 T cell receptor. No obvious explanation for the difference between the finite and continuous T lymphocyte cell lines was evident. One possible suggestion is that the finite T lymphocytes are polyclonal, whereas the continuous T cell lines are all monoclonal. It could be argued that the A7 construct is only stable expressed in combination with certain subfamilies of endogenous T cell receptors. For polyclonal finite T cell lines such a selection is possible, but this is not the case for clonal T cell lines: A T cell line with a clonal (endogenous) T cell receptor either allows a stable and functional expression of the A7 construct or it does not. The experiments performed thus questioned the validity of the concept depicted in FIG. 3 for cytotoxic continuous T cell lines.

Characterisation of the Melanoma Specific T Cell Line C-Cure 709

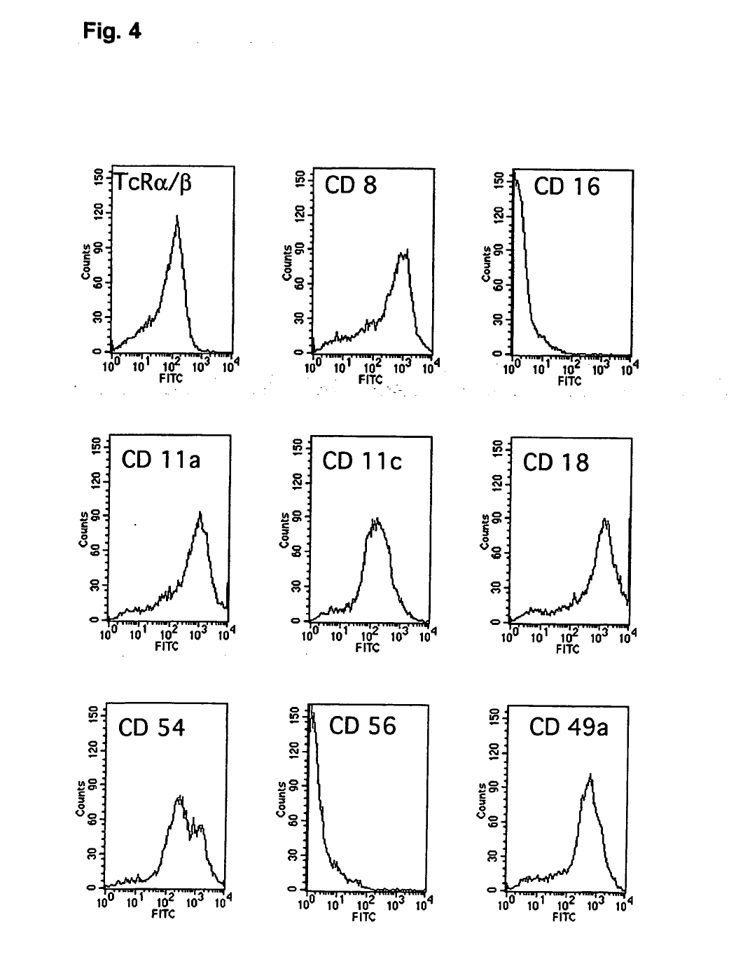

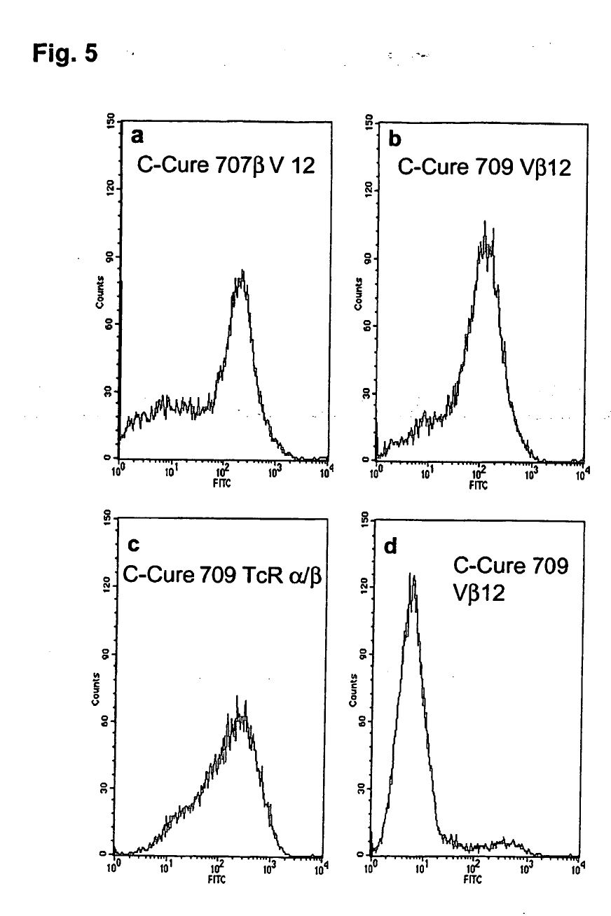

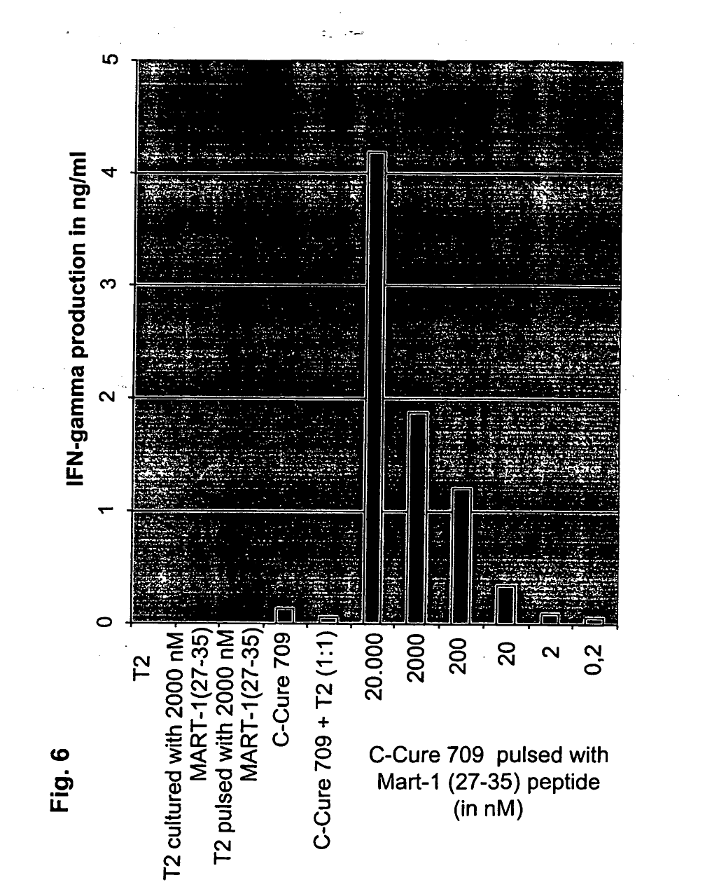

In the next series of experiments it turned out that the cytotoxic continuous T cell line C-Cure 707 could be stable transfected with the A7 construct leading to the melanoma specific cell line C-Cure 709. C-Cure 709 has so far shown stable expression of the A7 construct for more than 135 PD. The 1) phenotype (FIGS. 4 and 5), 2) specificity (FIG. 6) and 3) functional characteristics of C-Cure 709 (tables 2,3,4,5 and 9) is described in detail below.

1) C-Cure 707 is a normal (non-malignant) cytotoxic continuous T cell line established from TIL cells of a skin biopsy specimen from a patient with Sezary's syndrome in the presence of high concentrations of IL-2 and IL-4 (Kaltoft et al., 1998). Except for subfamily T cell receptor expression, C-Cure 707 and C-Cure 709 have a similar phenotype as shown by flow cytometry (FIG. 4) From the analysis it can be seen that both T cell lines express the TCR-2 (TCRα/β cell receptor). Also both cell lines express CD8+ that is expressed on cytotoxic T cells. CD 16 and CD56, markers expressed on natural killer (NK) cells, are absent on the two T cell lines. Both T cell lines have high expression of the protein complex CD11/CD18 that interacts with the adhesion protein known as ICAM-1 (CD54). CD54 is present on both T cell lines and it is known that most melanoma cell lines also express this protein. The appearance of CD49a on both T cell lines indicates previous activation of the cell lines.

The endogenous T cell receptor of C-Cure 707 as shown in FIG. 5(a) belongs to the V_{β}12 subfamily of the T cell receptor. Initially after A7 transfection C-Cure 709 also expressed V_{β}12 (FIG. 5b). Upon long term culture for more than 70 PD C-Cure 709 lost expression of V_{β}12 (FIG. 5d) while still retaining T cell receptor expression (FIG. 5c). The results indicate that the endogenous T cell receptor by long term culture of C-Cure 709 is downregulated, and the expression of V_{β}12 phenotypically distinguishes C-Cure 707 and C-Cure 709

2) In order to investigate whether C-Cure 709 specifically recognises Mart-1 (M9-2) in the context of HLA-A2, T2 cell were pulsed with different concentrations of Mart-1(M9-2) peptide and mixed with C-Cure 709 (10⁶/ml) at a 1:1 ratio in the presence of 2000 u/ml IL-2 and 500 u/ml IL-4. Interferon-γ (IFN-γ) production was measured after 20 hours as shown in FIG. 6. T2 is an HLA-A2+ TAP deficient B lymphoblastoid cell line. That T2 is TAP deficient means that it cannot transport endogenously synthesized peptides to its own HLA molecules. T2 can however bind exogeneously-added peptides like Mart-1 (M9-2). FIG. 6 shows that C-Cure 709 specifically recognises Mart-1 (M9-2) in the context of HLA-A2.

3) To test whether C-Cure 709 besides Mart-1 (M9-2) specificity also is capable of melanoma cell killing C-Cure 709 (effector cells) were mixed with the melanoma cell line (10⁵/ml) at different ratios as shown in table 2. Killing was monitored after 4 hours in a standard ⁵¹Cr release assay in a medium supplemented with 2000 u/ml IL-2 and 500 u/ml IL-4.

TABLE 2
Tumor cell killing within 4 hours of the 888-A2
melanoma cell line. The melanoma cell line 888 expressing
Mart-1 but not HLA-A2 is not killed by C-Cure 709 and the parental
C-Cure 707 does not kill 888-A2 (data not shown).

Effector:target ratio

| | 25:1 | 10:1 | 5:1 | 1:1 |
|---|---|---|---|---|
| Target cells 888-A2 | 65% | 45% | 30% | 15% |
| Target cells 888 | 5% | 6% | 4% | 3% |

Killing of the 888-A2 melanoma cell line is followed by cytokine production. The released cytokines have in the case of 888-A2 no tumoricidal (bystander) effect on the 888-A2 cell line. C-Cure 709 cultured in the presence of 2000 u/ml IL-2 and 500 u/ml IL-4 were washed and resuspended in fresh IL-2+IL-4 containing medium at a concentration of 10⁶/ml. Table 3 shows the induced and constitutive cytokine production in ng/ml after 20 hours. (interferon-γ (IFN-γ), tumour necrosis factor-α(TNF-α), granulocyte macrophage colony stimulating factor (GM-CSF), interleukin-(IL-5)) of C-Cure 709 with and without the addition of 888-A2 at an effector cell:target cell ratio of 3:1. The melanoma cell 888 does not increase the cytokine production of C-Cure 709 above the constitutive level and C-Cure 709 does not produce interleukin 10 (IL-10) (results not shown).

TABLE 3
Induced and constitutive cytokine production og C-Cure 709.

| | IFN-γ | TNF-α | GM-CSF | IL-5 |
|---|---|---|---|---|
| 888-A2 | 0.00 | 0.00 | 0.00 | 0.00 |
| C-Cure 709 | 0.83 | 0.00 | 5.28 | 0.89 |
| C-Cure 709 + 888- | 4.53 | 1.48 | 23.6 | 3.71 |
| A2 | | | | |

The C-Cure 709+T cell line used in table 3 was a month before the experiment shown, activated by Irradiated 888-A2 cells at a 3:1 ratio. This results in long-term constitutive IFN-γ, GM-CSF and IL-5 production by C-Cure 709. After activation of C-Cure 709 with 888-A2 the production of TNF-α ceases within a few hours whereas it takes weeks before IFN-γ, GM-CSF and IL-5 production reaches the level shown in table 3. This implies that C-Cure 709 has properties like activated/inflammatory T cells. Compared to TIL cells that are devoid of constitutive cytokine production, this property of C-Cure 709 may be of prime importance for the in vivo effect of C-Cure 709. Further experiments demonstrate that the constitutive and inducible cytokine production of C-Cure 709 changes over time and is dependent on and can be adjusted by the activation status of the T cell line (results not shown). The activation status of C-Cure 709 measured by cytokine production is critically dependent on the level of exogenous added IL-2 (or IL-15) Table 4 shown below shows a the cytokine profile of C-Cure 709 as a function of the concentration of IL-2.

TABLE 4
Influence of the concentration of IL-2 on the cytokine
production (measured after 20 hours) of C-Cure 709
(10⁶/ml) activated by 888-A2 at a 5:1 ratio

| | IFN-γ | TNF-α | IL-5 | GM-CSF |
|---|---|---|---|---|
| 2000 u/ml IL-2 | 6.3 | 1.05 | 4.5 | 19.6 |
| 1000 u/ml IL-2 | 3.7 | 0.35 | 2.9 | 10.0 |
| 100 u/ml IL-2 | 1.2 | 0.02 | 1.1 | 3.9 |
| 10 u/ml IL-2 | 0.62 | 0.00 | 0.37 | 3.3 |
| 1 u/ml IL-2 | 0.49 | 0.00 | 0.15 | 3.4 |
| 0 u/ml IL-2 | 0.47 | 0.00 | 0.12 | 3.1 |

Besides IL-2, the cytokine IL-15 can as shown in table 5 substitute IL-2 in the activation of C-Cure 709 stimulated with 888-A2 (at a 3:1 ratio) as shown in table 5. Cytokine production was measured after 20 hours.

TABLE 5
Cytokine production of C-Cure 709 (10⁶/ml) stimulated
with 888-A2 in the presence of IL-2 or IL-15. As IL-15 has a
broader tissue distribution than IL-2 the fact that IL-15 can
substitute for IL-2 may be of importance for the in vivo effects
of C-Cure 709. (IL-2 is only produced by activated T lymphocytes,
whereas epithelial cells, monocytes, macrophages
and dendritic cells produce IL-15).

| | IFN-γ | TNF-α | IL-5 | GM-CSF |
|---|---|---|---|---|
| no IL-2/IL-15 | 0.06 | 0.00 | 0.05 | 1.27 |
| no IL-2/IL15 + 888- | 1.9 | 0.56 | 0.27 | 11.5 |
| A2 | | | | |
| IL-2 | 0.23 | 0.00 | 0.23 | 8.4 |
| IL-2 + 888-A2 | 6.4 | 4.4 | 5.6 | 27 |
| IL-15 | 0.49 | 0.00 | 0.23 | 11.6 |
| IL-15 + 888-A2 | 8.27 | 3.1 | 6.6 | 29 |

### CONCLUSION

In conclusion the above mentioned experiments show that C-Cure 709 specifically recognises Mart-[(M9-2) restricted by HLA-A2 leading to melanoma cell killing followed by cyokine production. The insertion of just 2 genes in C-Cure 707 generating C-Cure 709, establishes a cell line that is capable of exploiting the complex cellular signal system leading to tumour cell killing concomitant with extensive cytokine production.

It is known that approximately 55% of Caucasians carry the HLA-A2 allele and that 90% of melanomas express Mart-1. It is thus expected that 50% of all patients suffering from malignant melanoma could have benefit of a treatment with C-Cure 709. For treatment it is the intention that C-Cure 709 is injected directly in the tumour. This is contrary to the TIL protocols, where the lymphocytes are given as a systemic treatment.

A day before injection of C-Cure 709, the cell culture is added 100 pM IL-12. As expected IL-12 increases the production of IFN-γ by C-Cure 709 (data not shown).

Before injection C-Cure 709 is resuspended in serum from the patient and lethally γ-irradiated (60Gy) preventing C-Cure 709 from cell division. The irradiation has only a minor influence on the killing ability and cytokine production by C-Cure 709 during the first day or two (data not shown). After this time C-Cure 709 will die due to the effect of the irradiation.

### Example 2

Presentation of Superantigens on Tumour Target Cells

Superantigen are not real antigens in the sense that they bind MHC class II without being processed. Staphylococcus aureus and streptococci produce a large family of exotoxins, which encompass staphylococcal enterotoxins (SE) and the group streptococcal pyrogenic exotoxins. These proteins are prototypic superantigens: they i) bind with mediate/high affinity to HLA class II molecules, ii) are presented to T cells by antigen presenting cells (APC) in a HLA class II-dependent but not HLA class II-restricted manner, iii) stimulate large populations of T cells expressing particular T cell receptor β chain variable segments (subfamily segments). A number of different SE's are known and purified, such as staphylococcus A,B,C,D,E and H (SEA, SEB, SEC, SED, SEE and SEH).

The generation of continuous tumour specific T lymphocyte cell lines exemplified above redirects the specificity of the T cells by insertion of genes encoding specificity. However, T cells can also be targeted to tumour cells by external means.

As exemplified below targeting superantigens to tumour cells can activate T cells to a strong immune attack directed against the malignant cells. This attack is similar to the T cell receptor approach in that it mediates cytotoxicity towards the tumour cells followed by cytokine/chemokine production leading to collateral tumour destruction.

Multivalent presentation of superantigen on tumour target cells will promote efficient T cell activation such as cell killing and cytokine production. Although there are far more T-lymphocytes in the body responding to superantigens than to tumour antigens a systemic approach also suffers from the lack of an insufficient amount of effector cells, as the effector cells commit suicide or become anergic upon encounter with the superantigen. Moreover many normal cells like monocytes/macrophages, dendritic cells, B cells express HLA class II antigens, that are receptors for superantigens. Destroying these normal and vital immune cells are expected to have severe side effects. However, the advantage of superantigen mediated tumour cell killing is that they are the most potent activators of T-lymphocytes known.

Compared to tumour antigens that often is self-antigens and hence rather weak antigens, superantigens are superior in mediating activation of T cells. For instance, C-Cure 703 that transiently expressed the transferred A7 TCR mentioned in example 1 is not capable of producing IL-2 upon encounter with melanoma cells. However, large amounts of IL-2 are produced, when C-Cure 703/A7 (which is HLA class II positive) is stimulated with SEA (table 6). If this is representative for the in vivo situation, systemic IL-2 treatment might be avoided.

TABLE 6
Table 6 shows IL-2 production (in ng/ml) of the cell lines
C-Cure 703 and C-Cure 703 transfected with the A7 TCR
construct (C-Cure 703/A7). The T cell lines were stimulated
with 888-A2 (at a 10:1 ratio with 10⁶ lymphocytes/ml)
compared with the same concentration of T lymphocytes
stimulated with 500 ng/ml SEA for 20 hrs.

| Stimulant | 888-A2 | SEA |
|---|---|---|
| C-Cure 703 | 0.00 | 0.00 |
| C-Cure 703/A7 | 0.00 | 2.63 |

Besides IL-2, SEA, stimulation produces IFN-γ, TNF-α, GM-CSF, IL-4 and IL-5. As shown in table 6 C-Cure. 703/A7 mediate efficient tumour cell killing of HLA class II positive SEA pulsed tumour cells, like Daudi and Se-Ax. (Daudi is a Burkitt lymphoma B cell line and Se-Ax is a leukemic T cell line established from a patient with Sezary's syndrome). HLA class II antigens are moderate/high affinity receptors for superantigens. Binding of superantigens to HLA class II positive cells activates cytotoxic T lymphocytes to mediate killing of the target cells. Cell killing by C-Cure 703/A7 of target cells in the presence of 500 ng/ml SEA in 3 hours is shown in table 7.

TABLE 7

Effector cell:target ratio

| | 40:1 | 20:1 | 10:1 | 5:1 |
|---|---|---|---|---|
| Daudi | 78% | 55% | 39% | 22% |
| Se-Ax | 67% | 42% | 30% | 14% |

The V_{β}22 TCR expressing continuous T lymphocyte cell line C-Cure 702 responds to SEA in a similar fashion as C-Cure 703/A7, because V_{β}22 is a SEA responsive element. Superantigen mediated killing is not HLA restricted. This implies that allogeneic continuous T lymphocytes can be used for adjuvant superantigen mediated therapy irrespective of HLA type.

The data shown above applies to tumour cells expressing HLA class II antigens as HLA class II expression directs the superantigen to the tumour cell. As mentioned above however many normal cells also express HLA class II antigens. Natural superantigen treatment of HLA class II positive tumour cells as such are thus expected, besides tumour cell killing, to result in severe side effects. However, HLA class II negative (and HLA class II positive tumour cell lines) can be efficiently coated with superantigens, if tumour specific antibodies or their Fab/scFv fragments are available, see description of chimeric receptors herein above. Such antibodies are covalently coupled to superantigens thus targeting superantigens to tumour cells (Tumour targeted superantigens, TTS) (Tordsson et al., 2000). The antibody/superantigen constructs mediate efficient tumour cell elimination (Tordsson et al., 2000). To avoid binding of superantigen to normal HLA class II positive cells mutations in the HLA binding site(s) of the superantigen can be introduced (Tordsson et al., 2000). Such mutations do not affect T cell receptor binding with subsequent T cell activation (Tordsson et al., 2000). However, side effects resulting from superantigen binding to normal cells are reduced. The side effects of using such TTS constructs are expected to be minimal compared to natural superantigens.

This TTS approach can also be combined with T cell receptor approach as exemplified by the melanoma example above. The C-Cure 709 melanoma specific continuous T cell line expresses V_{β}7.3, a SEA responsive element.

The consequence of introducing the A7 vector into C-Cure 707 is illustrated below. The cytokine profile upon activation with several superantigens of the basic-Cure 707 T cell line is shown in table 8. 10⁶ cells/ml were stimulated for 20 hours with the following superantigens: SEA, SEB, SEC1, SED and SEE (each 1 ng/ml) in a medium-containing 2000 u/ml IL-2 and 500 u/ml IL-4. The cytokine concentrations are in ng/ml.

TABLE 8

| | IFN-γ | TNF-α | IL-10 | GM-CSF |
|---|---|---|---|---|
| No | 0.70 | 0.00 | 0.00 | 0.60 |
| addition | | | | |
| SEA | 0.74 | 0.00 | 0.00 | 0.83 |
| SEB | 4.40 | 1.00 | 0.00 | 4.58 |
| SEC | 12.5 | >25. | 0.00 | 23.8 |
| SED | 1.57 | 0.24 | 0.00 | 1.66 |
| SEE | 0.79 | 0.00 | 0.00 | 0.90 |

As shown in table 9 C-Cure 707 respond well to SEC, in agreement with the fact the C-Cure 707 expresses V_{β}12, a SEC responsive element. It is also evident from table 9 that C-Cure 707 does not (or only very weakly) respond to SEA (and SEE).

When C-Cure 709 also expressed V_{β}12 a strong response towards SEC was observed (not shown). As V_{β}12 during continuous culture was lost (fFIG. 3) C-Cure 709 only weakly responded to SEC (not shown). However contrary to C-Cure 707, Cure 709 responds strongly to SEA confirming the transfer of the SEA responsive element A7 (table 9). Cure 709 was stimulated with SEA similar to C-Cure 707 depicted in table 8.

TABLE 9

| | IFN-γ | TNF-α | IL-10 | GM-CSF |
|---|---|---|---|---|
| No | 1.38 | 0.00 | 0.00 | 1.58 |
| addition | | | | |
| SEA | 8.04 | 5.44 | 0.00 | 17.9 |

SEA can be coupled to the anti-high molecular melanoma associated antigen antibody K305 (or antibodies with similar specificity). Such a constuct is expected to give C-Cure 709 a bi-specific weapon against the melanoma cells: The Mart-1 specific T cell receptor A7 and the activation of C-Cure 709 by SEA via binding of K305 to melanoma cells. It is furthermore expected that the SEA coupled antibody fragment when reaching the tumour will activate resident immune T cells to further cytokine/chemokine production.

The above-suggested approach should greatly reduce the risk of tumour escape.

IL-2 and IL-15 and similar cytokines can also be combined with tumour specific antibodies Such constructs are expected to localize to tumour areas, where they could activate injected continuous T cell lines as well as resident T lymphocytes similar to the T cell receptor and TTS approach.

Treatment of many cancers may benefit from the strategy outlined above. The application of continuous tumour specific T cell may be combined with proven treatment regiments, such as surgery, chemotherapy and radiation therapy. Applications of tumour specific continuous T cell lines may also be combined with other immunotherapeutic protocols such as autologous TIL treatment and dendritic cell vaccination.

As methods are available to generate a number of continuous T cell lines it may in time be possible to generate a bank of universal allogeneic tumour specific continuous T cell clones that may fit most HLA types patients can possess. The unlimited amount of tumour specific T cells that can be made from a continuous T cell line, should greatly facilitate systemic studies of T cell trafficking to tumour tissue and should aid our understanding of how the immune system can be turned against tumours.

### REFERENCES

Abken H, Hombach A, Reinhold U, Ferrone S. Can combined T-lymphocyte and antibody-based-immunotherapy outsmart tumour cells?Immunol Today 1998; 192-5.

Altenschmidt U, Klundt E, Groner B. Adoptive transfer of in vitro-targeted, activated T lymphocytes results in total tumour regression. J. Immunol. 1997; 159:5509-15

Anichini A, Maccalli C, Mortarini R, Salvi S, Mazzocchi A, Squarcina P, Herlyn M, Parmiani G. Melanoma cells and normal melanocytes share antigens recognized by HLA-A2-restricted cytotoxic T cell clones from melanoma patients. J Exp Med. 1993;177(4):989-98.

Arienti F, Belli F, Rivoltini L, Gambacorti-Passerini C, Furlan L, Mascheroni L et al. Adoptive immunotherapy of advanced melanoma patients with interleukin-2 (IL-2) and tumour-infiltrating lymphocytes selected in vitro with low doses of IL-2. Cancer Immunol Immunother 1993; 36:315-322.

Barth R J, Mule J J, Spiess P J, Rosenberg S A. Interferon gamma and tumor necrosis factor have a role in tumor regressions mediated by murine CD8+ tumor-infiltrating lymphocytes. J Exp Med. 1991;173(3):647-58.

Barth A, Wanek L A, Morton D L. Prognostic factors in 1,521 melanoma patients with distant metastases [see comments]. J Am Coll Surg 1995; 18:193-201.

Clay T M, Custer M C, Sachs J, Hwu P, Rosenberg S A, Nishimura Ml. Efficient transfer of a tumour antigen-reactive TCR to human peripheral blood lymphocytes confers anti-tumour reactivity. J Immunol 1999; 163:507-513.

Dall P, Hekele A, Beckmann M W, Bender H G, Herrlich P, Ponta. Efficient lysis of CD44v7/8-presenting target cells by genetically engineered cytotoxic T-lymphocytes -a model of immunogene therapy of cervical cancer. Gynecel. Oncol. 1997; 66:209-16 Dunbar P R, Chen J L, Chao D, Rust N, Teisserenc H, Ogg G S et al. Cutting edge: rapid cloning of tumour-specific CTL suitable for adoptive immunotherapy of melanoma. J Immunol 1999; 162:6959-6962.

Goedegebuure P S, Douville L M, Li H, Richmond G C, Schbof D D, Scavone M et al. Adoptive immunotherapy with tumour-infiltrating lymphocytes and interleukin-2 in patients with metastatic malignant melanoma and renal cell carcinoma: a pilot study. J Clin Oncol 1995; 13:1939-1949.

Hung K, Hayashi R, Lafond-Walker A, Lowenstein C, Pardoll D, Levitsky H. The central role of CD4(+) T cells in the antitumour immune response. J Exp Med 1998; 188:2357-2368.

Kaltoft K, Bisballe S, Dyrberg T, Boel E, Rasmussen P B, Thestrup-Pedersen K. Establishment of two continuous T-lymphocyte strains from a single plaque of a patient with mycosis fungoides. In Vitro Cell Dev Biol 1992; 28A:161-167.

Kaltoft K, Pedersen C B, Hansen B H, Lemonidis A S, Frydenberg J, Thestrup-Pedersen K. In vitro genetically aberrant T-lymphocyte clones with continuous growth are associated with atopic dermatitis. Arch Dermatol Res 1994; 287:4247.

Kaltoft K, Pedersen C B, Hansen B H, Thestrup-Pedersen K. Appearance of isochromosome 18q can be associated with in vitro immortalization of human T lymphocytes. Cancer Genet Cytogenet 1995; 81:13-16.

Kaltoft K, Hansen B H, Pedersen C B, Pedersen S, Thestrup-Pedersen K Common clonal chromosome aberrations in cytokine-dependent continuous human T-lymphocyte cell lines. Cancer Genet Cytogenet 1995; 85:68-71.

Kaltoft K. Cytokine-driven immortalization of in vitro activated human T lymphocytes. CD28 expression correlates inversely with cell population doublings. Exp Clin Immunogenet 1998; 15:84-89.

Kawakami Y, Nishimura M I, Restifo N P, Topalian S L, O'Neil B H, Shilyansky J. Yannelli J R, Rosenberg S A T-cell recognition of human melanoma antigens. J Immunother. 1993;14(2):88-93

Kessels H W H G, Wolkers M C, van den Boom M D, van den Valk M A and Schumacher T N N. Immunotherapy through TCR gene transfer. Nature Immunology 2, 957-961, 2001.

Kugler A, Stuhler G, Walden P, Zoller G, Zobywalski A, Brossart P et al. Regression of human metastatic renal cell carcinoma after vaccination with tumour cell-dendritic cell hybrids [see comments]. Nat Med 2000; 6:332-336.

Lenardo M J, Interleukin-2 programs mouse alpha beta T lymphocytes for apoptosis. Nature. 1991; 31;353(6347):858-61.

Liu X, Peralta, E A, Ellenhorn J D I, Diamond D J., Targeting of human p53-overexpressing tumour cells by an HLA A 0201-restricted murine T-cell receptor expressed in Jurkat T-lymphocytes. Cancer Research. 2000; 60:693-70

Liu K and Rosenberg S A. Transduction of an IL-2 gene into human melanoma-reactive lymphocytes results In their continued growth in the absence of exogeneous IL-2 and maintenance of specific antitumor activity. J immunol 167. 6356-6365, 2001.

McInemey J M, Nawrocki J R and Lowrey C H., Long-term silencing of retroviral vectors is resistant to reversal by trichostatin A and 5-azacytidine. Gene Therapy 7, 653-663, 2000.

Nestle F O, Alijagic S, Gilliet M, Sun Y, Grabbe S, Dummer R et al. Vaccination of melanoma patients with peptide- or tumour lysate-pulsed dendritic cells [see comments]. Nat Med 1998; 4:328-332.

Perillo N L, Watford R L, Newman M A, Effros R B. Human T lymphocytes possess a limited in vitro life span. Exp Gerontol 1989; 24:177-187.

Queirolo P, Ponte M, Gipponi M, Cafiero F, Peressini A, Semino C et al. Adoptive immunotherapy with tumour-infiltrating lymphocytes and subcutaneous recombinant interieukin-2 plus interferon alfa-2a for melanoma patients with nonresectable distant disease: a phase Ill pilot trial. Melanoma Istituto Scientifico Tumouri Group. Ann Surg Oncol 1999; 6:272-278.

Rosenberg S A, Yannelli J R, Yang J C, Topalian S L, Schwartzentruber D J, Weber J S et al. Treatment of patients with metastatic melanoma with autologous tumour-infiltrating lymphocytes and interleukin 2. J Natl Cancer Inst 1994; 86:1159-1166

Rosenberg S A, White D E, Vitiligo in patients with melanoma: normal tissue antigens can be targets for cancer immunotherapy. J Immunother Emphasis Tumor Immunol 1996; 19:81-84

Rossig C, Bollard C M, Nuchtem J G, Merchant D A and Brenner Mk. Targetting of G(D2)-positive tumor by human T lymphocytes engineered to express chimeric T-cell receptor genes. Int J Cancer 94(2) 228-236, 2001.

Schwartzentruber D J, Horn S S, Dadmarz R, White D E, Yannelli J R, Steinberg S M et al. In vitro predictors of therapeutic response in melanoma patients receiving tumour-infiltrating lymphocytes and interleukin-2. J Clin Oncol 1994; 12:1475-1483.

Shi Y, Zheng W and Rock K L. Cell injury releases endogenous adjuvant that stimulate cytotoxic T cell responses. Proc. Natl. Acad. Sci. 2000; 97:14590-14595

Soiffer R, Lynch T, Mihm M et al. Vaccination with irradiated autologous melanoma cells engineered to secrete human granulocyte-macrophage colony-stimulating factor generates potent antitumour immunity in patients with metastatic melanoma. PNAS 1998:95 13141-13146.

Tordsson J M, Ohlsson L G, Abrahmsen L B, Karlstrom P J, Lando P A, Brodin T N. Phage-selected primate antibodies fused to superantigens for immunotherapy of malignant melanoma. Cancer Immunol Immunother 2000; 48 691-702.

Trefzer U, Weingart G, Chen Y, Herberth G, Adrian K, Winter H et al. Hybrid cell vaccination for cancer immune therapy: first clinical trial with metastatic melanoma. Int J Cancer 2000; 85:618-626.

Visonneau S, Cesano A, Porter D L, Luger S L, Schuchter L, Kamoun M et al. Phase I trial of TALL-104 cells in patients with refractory metastaic breast cancer (In Process Citation]. Clin Cancer Res 2000; 6:1744-1754. P Zaks T Z, Chappell D B, Rosenberg S A, Restifo N P. Fas-mediated suicide of tumour-reactive T cells following activation by specific tumour: selective rescue by caspase inhibition. J Immunol 1999; 162:3273-3279.

1. A continuously growing, normal, human T-lymphocyte cell line, capable of undergoing at least 30 population doublings in vitro and capable of activation at least once, wherein said T-lymphocyte cell line comprises an antigen specific immune receptor encoded by at least one first nucleotide sequence operably linked to a second nucleotide sequence comprising an expression signal not natively associated with the first nucleotide sequence.

2. The T-lymphocyte cell line according to claim 1, comprising first nucleotide sequences encoding an antigen specific T-cell receptor.

3. The T-lymphocyte cell line according to claim 1, comprising first nucleotide sequences encoding the Variable-Joining sequences of an a chain and/or Variable-Diversity-Joining sequences of ass chain of an antigen specific T-cell receptor.

4. The T-lymphocyte cell line according to claim 1, comprising first nucleotide sequences encoding chimeric immune receptors consisting of a fusion between a T-lymphocyte signaling unit with a specific antibody binding part recognising a specific antigen.

5. The T-lymphocyte cell line according to claim 1, comprising first nucleotide sequences encoding chimeric immune receptors consisting of a fusion between a 4 chain of CD3 with a single-chain antibody recognising a specific antigen

6. The T-lymphocyte cell line according to claim 1, comprising first nucleotide sequences encoding tumour specific antibodies coupled to or fused with sequences encoding one or more cytokines.

7. The T-lymphocyte cell line according to claim 1, wherein the antigen specific immune receptor recognises an antigen in the context of an MHCclass I molecule.

8. The T-lymphocyte cell line according to claim 1, wherein the antigen specific immune receptor recognises an antigen in the context of an HLA-A2 molecule.

9. The T-lymphocyte cell line according to claim 1, wherein the antigen specific immune receptor recognises an antigen in the context of an MHC class II molecule.

10. The T-lymphocyte cell line according to claim 1, wherein the antigen specific immune receptor recognises a tumour associated antigen.

11. The T-lymphocyte cell line according to claim 1, wherein the antigen specific immune receptor recognises a melanoma associated antigen.

12. The T-lymphocyte cell line according to claim 1, wherein the antigen specific immune receptor recognises a melanoma associated antigen selected from the group consisting of tyrosinase antigens, gp100 antigens or MART-1 antigens.

13. The T-lymphocyte cell line according to claim 1, wherein the antigen specific immune receptor recognises a tyrosinase antigen comprising the amino acid sequence MLLAVLYCL.

14. The T-lymphocyte cell line according to claim 1, wherein the antigen specific immune receptor recognises a gp100 antigen comprising the amino acid sequence KTWGQYWQV.

15. The T-lymphocyte cell line according to claim 1, wherein the antigen specific immune receptor recognises a MART-1 antigen.

16. The T-lymphocyte cell line according to claim 1, wherein the antigen specific immune receptor recognises a MART-1 antigen selected from the group consisting of:AAGIGILTV, EAAGIGILTV and AAGIGILTVI.

17. The T-lymphocyte cell line according to claim 1, wherein the chain of the T-cell receptor is encoded by nucleic acid sequences selected from the group consisting of (a) Va8.2/Jα49/Ca chain and V 13. 6/Dp1.1/Jss1. 5/C respectively (b) Va17/Jα42/Ca and Vus6. 5/Dss0.1/Jss0.5/Css; (c) Va9/Ja16/Ca and Vss22. 1/Dss2. 1/Jss2. 1/Css2; (d) Val/Jα49/Ca and Vss7. 3/Dss2.6/Jss2.1/Css2; (e) Va25/Jα54/Ca and Vss3. 1/Dss1. 1/Jp1. 1/Css1; (f) Vα21/Jα42/Ca and Vss7.3/Dss2.1/Jss2.7/Css2; and (g) nucleic acid sequences encoding a T-cell receptor which retain the antigen recognition function of said T-cell receptor encoded by (a)-(f).

18. The T-lymphocyte cell line according to claim 1, which is capable of undergoing at least 50 PD.

19. The T-lymphocyte cell line according to claim 1, which is capable of undergoing at least 75 PD.

20. The T-lymphocyte cell line according to claim 1, which is capable of undergoing at least 100 PD.

21. The T-lymphocyte cell line according to claim 1, which is capable of undergoing at least 150 PD.

22. The T-lymphocyte cell line according to claim 1, which is derived from tumour infiltrating lymphocytes (TILs).

23. The T-lymphocyte cell line according to claim 1, which originates from a biopsy taken at the site of disease.

24. The T-lymphocyte cell line according to claim 1, which is derived from a body fluid.

25. The T-lymphocyte cell line according to claim 1, which is derived from a tissue sample.

26. The T-lymphocyte cell line according to claim 1, which is derived from a patient with Sezary's syndrome.

27. The T-lymphocyte cell line according to claim 1, which is derived from a skin biopsy from a patient with sezary's syndrome.

28. The T-lymphocyte cell line according to claim 1, wherein the T-lymphocytes are selected from the group consisting of CD4+ T-lymphocytes, CD8+ T lymphocytes and CD4-/CD8-T-lymphocytes.

29. The T-lymphocyte cell line according to claim 1, wherein the T-lymphocytes are selected from the group consisting of inflammatory T-lymphocytes, cytotoxic T lymphocytes, regulatory T-lymphocytes and helper T-lymphocytes.

30. The T-lymphocyte cell line according to claim 1, which is a CD8+ T-lymphocyte cell line

31. The T-lymphocyte cell line according to claim 1, which is a cytotoxic T lymphocyte line.

32. The T-lymphocyte cell line according to claim 1, which is a cytotoxic T lymphocyte line, with tumour cell killing activity.

33. The T-lymphocyte cell line according to claim 1, which can be activated at least once.

34. The T-lymphocyte cell line according to claim 1, which is disease activated.

35. The T-lymphocyte cell line according to claim 1, which can secrete one or more cytokines selected from:IFN-γ, IL-10, TNF-a, IL-12, IL-2,IL-4, IL-5, IL-18, IL-21 and/or IFN-γ and/or GM-CSF.

36. The T-lymphocyte cell line according to claim 1, which can secrete IL-5.

37. The T-lymphocyte cell line according to claim 1, which secretes between 0.5 and 10 ng/ml/10⁶ cells/20 hours IL-5, following activation.

38. The T-lymphocyte cell line according to claim 1, which can secrete GM-CSF.

39. The T-lymphocyte cell line according to claim 1, which secretes between 5 and 50 ng/ml/10⁶ cells/20 hours GM-CSF, following activation.

40. The T-lymphocyte cell line according to claim 1, which can secrete IFN-gamma.

41. The T-lymphocyte cell line according to claim 1, which secretes between 0.5 and 10 ng/ml/10⁶ cells/20 hours IFN-gamma, following activation.

42. The T-lymphocyte cell line according to claim 1, which can secrete TNF-a.

43. The T-lymphocyte cell line according to claim 1, which secretes between 0.5 and 10 ng/ml/10⁶ cells/20 hours TNF-α, following activation.

44. The T-lymphocyte cell line according to claim 1, which secretes two or more cytokines selected from the group consisting of: IL-5, CM-CSF, IFN-γ and/or TNF-a according to claim 37,39,41 and 43.

45. The T-lymphocyte cell line according to claim 1, which are cultured in the presence of at least two factors that promote T-lymphocyte growth.

46. The T-lymphocyte cell line according to claim 1, which are cultured in the presence of IL-2 and IL-4.

47. The T-lymphocyte cell line according to claim 1, which are cultured in the presence of at least 1 nM IL-2.

48. The T-lymphocyte cell line according to claim 1, which are cultured in the presence of at least 1 nM IL-4.

49. The T-lymphocyte cell line according to claim 1, which has been cultured in the presence of at least 10 pM IL-12.

50. The lymphocyte cell line according to claim 1, where the nucleic acids encoding said antigen specific immune receptor are comprised within an expression vector.

51. The lymphocyte cell line according to claim 1, where the nucleic acids encoding said antigen specific immune receptor are comprised within a viral expression vector or a vector which is derived from a virus.

52. The lymphocyte cell line according to claim 1, where the nucleic acids encoding said antigen specific immune receptor are comprised within an expression vector which is of retroviral origin.

53. The lymphocyte cell line according to claim 1, where the nucleic acids encoding said expression signals are derived from a prokaryotic, a eukaryotic, a viral or a plasmid source.

54. The lymphocyte cell line according to claim 1, where the nucleic acids encoding said expression signals are selected from the group consisting of Moline murine leukemia virus long terminal repeat and a hybridHTLV-I/SV40 SRa promoter.

55. The lymphocyte cell line according to claim 1, where the nucleic acids encoding said antigen specific immune receptor have been introduced by retroviral transfer.

56. (Cancelled)

57. A pharmaceutical composition comprising at least one T-lymphocyte cell line according to claim 1.

58. The pharmaceutical composition according to claim 57, wherein the T lymphocytes have been attenuated prior to administration.

59. The pharmaceutical composition according to claim 57, wherein the T lymphocytes have been attenuated by γ-irradiation prior to administration.

60. The pharmaceutical composition according to claim 57 and further comprising a caspase inhibitor.

61. A method for treatment of an individual, said method comprising the steps of: i) Providing a T-lymphocyte cell line according to claim 1 ii) Providing an individual in need of treatment with said T lymphocyte cell line iii) Treating said individual by administering to said individual a pharmaceutical effective amount of said T-lymphocyte cell line.

62. The method according to claim 61, wherein said treatment is prophylactic, curative or ameliorating.

63. The method according to claim 61, wherein said treatment is allogeneic immunotherapy.

64. The method according to claim 61, wherein said individual is diagnosed as suffering from cancer or a viral infection.

65. The method according to claim 61 wherein said individual is diagnosed as suffering from a cancerous disease selected from the group consisting of malignant melanoma, renal carcinoma, breast cancer, lung cancer, cancer of the uterus, prostatic cancer, lymphom, leukemia, cutaneous lymphom, hepatic carcinoma, colorectal cancer and sarcoma.

66. The method according to claim 61 wherein the step iii) further comprise treating said individual with one or more second therapies against cancer which could be selected from the group consisting of surgical treatment, chemotherapy, radiation therapy, therapy with cytokines, Hormone therapy, gene therapy, dendritic cell therapy and treatments using laser light.

67. The method according to claim 66, wherein said second therapy is dendritic cell therapy.

68. The method according to claim 66, wherein said second therapy is dendritic cell therapy, wherein the dendritic cells comprises nucleic acid sequences introduced by genetic manupulation.

69. The method according to claim 66, wherein said second therapy is dendritic cell therapy, wherein the dendritic cells comprises nucleic acid sequences encoding human tyrosinase.

70. The method according to claim 61, wherein step iii) further comprises administration of an inhibitor of activation induced cell death (AICD), either simultanously or sequentially.

71. The method according to claim 61, wherein step iii) comprise administering 10⁵-10¹² of said T-lymphocytes per dose.

72. The method according to claim 61 wherein step iii) comprise administering said T-lymphocyte cell line as a single dose.

73. The method according to claim 61, wherein step iii) comprise administering said T-lymphocyte cell line as more than one dose.

74. The method according to claim 61, wherein step iii) comprise administering said T-lymphocyte cell line parenterally.

75. The method according to claim 61, wherein step iii) comprise administering said T-lymphocyte cell line by injection directly into a tumour.

76. (Cancelled)

77. (Cancelled)

78. (Cancelled)

79. A method of constructing the T-lymphocyte cell line according to claim 1, said method comprising the step of: i) Introducing at least one first nucleotide sequence encoding an antigen specific immune receptor operably linked to a second nucleotide sequence comprising an expression signal not natively associated with the first nucleotide sequence into a continuously growing, normal
T-lymphocyte cell line, capable of undergoing at least 30 population doublings in vitro and capable of activation at least once.

80. A method of cultivating the T-lymphocyte cell line according to claim 1, said method comprising the steps of: i) Providing said T-lymphocyte cell line ii) Cultivating said T-lymphocyte cell line under conditions allowing expression of the antigen specific immune receptor

81. The method according to 80, wherein said conditions comprise the presence of IL-2 and IL-4.

82. The method according to 80, wherein said conditions comprise the presence of at least 1 nM IL-2.

83. The method according to 80, wherein said conditions comprise the presence of at least 1 nM IL-2.
